(19) United States
(12) Patent Application Publication
(10) Pub. No.: US 2023/0175019 A1
(43) Pub. Date: Jun. 8, 2023

(54) SCALABLE TRIO GUIDE RNA APPROACH FOR INTEGRATION OF LARGE DONOR DNA

(71) Applicant: University of Southern California, Los Angeles, CA (US)

(72) Inventors: Scott E. Fraser, Los Angeles, CA (US); Le Trinh, Los Angeles, CA (US)

(73) Assignee: University of Southern California, Los Angeles, CA (US)

(21) Appl. No.: 17/927,454

(22) PCT Filed: May 28, 2021
(86) PCT No.: PCT/US2021/034984

§ 371 Date: Nov. 23, 2022

Related U.S. Application Data
(60) Provisional application No. 63/031,203, filed on May 28, 2020.

(51) Int. Cl. C12N 15/90; A01K 67/027; C12N 15/113; C12N 15/65; C12N 15/79

(52) U.S. Cl. CPC C12N 15/902; A01K 67/0275; C12N 15/113; C12N 15/65; C12N 15/79; A01K 2217/072; A01K 2227/40; A01K 2267/03; C12N 2310/20; C12N 9/22

(57) ABSTRACT

A new DNA knock-in approach is provided based on the usage of three single guide RNA (sgRNA) to increase the integration efficiency of donor DNA based on the CRISRP-Cas system. The approach uses a pair of universal sgRNAs complementary to the donor DNA and a single sgRNA that targets the locus of interest. In various embodiments, targeting is achieved by pre-forming a DNA:RNA:protein (DNA:RNP) complex in vitro and introducing the complex into the embryo or cells of interest either by microinjection or transfection.

### CROSS-REFERENCE TO RELATED APPLICATIONS

This application includes a claim of priority under 35 U.S.C. § 119(e) to U.S. provisional patent application No. 63/031,203, filed May 28, 2020, the entirety of which is hereby incorporated by reference.

### REFERENCE TO SEQUENCE LISTING

The Sequence Listing submitted May 28, 2021 as a text file named “SequenceListing-065715-000108WO00_ST25” created on May 27, 2021 and having a size of 4,427 bytes, is hereby incorporated by reference.

### TECHNICAL FIELD

The present invention relates to gene editing using RNA-guided nucleases.

### BACKGROUND

The CRISPR-Cas system has emerged as a powerful molecular tool for genome editing including the tagging of endogenous loci. The system consists of a dual-RNA structure containing a CRISPR RNA (crRNA) and a trans-activating CRISPR RNA (tracrRNA) that has been engineered into a single-guide RNA (sgRNA) that associates with the Cas9 endonuclease to form a ribonucleoprotein (RNP) complex (sgRNA+Cas9 protein) capable of double-stranded DNA recognition and cleavage. The DNA targeting of the RNP complex is driven by about 20 nucleotides of complementary RNA-DNA base-pairing between the sgRNA and the target DNA. Essential to RNA-DNA recognition is the protospacer adjacent motif (PAM), a three nucleotide sequence (5′NGG-3′), adjacent to the DNA recognition site on the non-complement strand.

The simple rules used by the CRISPR-Cas system for sequence recognition and cleavage of double-stranded DNA has allowed for targeted manipulation of the genome. The CRISPR-Cas system can be used for mutagenesis or delivery of exogenous DNA using the non-homology end joining repair (NHEJ) or homology directed repair (HDR) pathways in cells. For mutagenesis, targeting of genomic sequence by sgRNA-Cas9 complex leads to double-stranded DNA breaks that the cell repairs via the NHEJ pathway. This process is highly efficient in eukaryotic cells and has been used extensively to create mutant alleles for study of gene function. While DNA mutagenesis using the non-homology end joining repair (NHEJ) pathway by CRISPR-Cas is highly efficient, targeted delivery of donor DNA by homology directed repair (HDR) or NHEJ is very inefficient.

Several groups have reported the targeting of endogenous proteins via the CRISPR-Cas systems to knock-in DNA oligos or GFP reporters. However, these approaches rely on HDR and observe drastic decrease of insertional efficiency (<1%) when the size of the donor DNA is increased beyond 200 bp. These inefficiencies limit the size of the donor DNA that can be delivered. The size limit for donor DNA presents a major disadvantage in protein tagging, whereas small protein tags (e.g. HA or His) require immunostaining to detect the tag which precludes live imaging studies. While fluorescent protein (FP) tagging allows for live imaging, coding sequences for FPs range in the 700-1000 bp size. These DNA sizes present a challenge for integration via the CRISPR-Cas system.

Therefore, it is an objective of the present invention to provide a system for efficient integration of exogenous DNA into a target genome, especially a large size exogenous DNA to detectably tag a genome protein without adversely affecting the transcription or expression of the genome protein.

It is another objective of the present invention to provide a method of genomic editing, especially gene knock-in, with increased efficiency.

### SUMMARY OF THE INVENTION

Various embodiments provide systems for integrating a donor sequence into a target locus, wherein the systems comprise (at least) three guide RNA (gRNA), wherein the first and second gRNA are capable of binding upstream and downstream of the donor sequence, respectively, thereby flanking the donor sequence, and the third gRNA is capable of binding a target locus. Generally by being capable of binding a DNA substrate at a locus, gRNA comprises a sequence complementary to the locus of the DNA substrate; and gRNA recruits or is capable of guiding a RNA-guided nuclease to the locus. In various embodiments, the first and second gRNA can flank the donor sequence and are independently capable of guiding a nuclease to a respective region of the donor sequence flanked by the first and second gRNA, and the third gRNA can bind a locus of interest in the genome of the target cell and guide a nuclease (same or different compared to the nuclease guided by the first and second gRNA) to the bound locus of interest by the third gRNA. In various embodiments, an RNA-guided nuclease can be a Cas nuclease.

Some embodiments provide that the systems further include the donor sequence in a nucleic acid vector and a quantity of a Cas nuclease, in addition to the three gRNA. In some aspects, the system can include a first complex formed from the donor sequence, the first gRNA, and a Cas nuclease; a second complex formed from the donor sequence, the second gRNA, and a Cas nuclease; as well as a third complex formed from the third gRNA and a Cas nuclease. In various aspects, the donor sequence is in a plasmid. In other aspects, the donor sequence is linearized DNA.

In various embodiments, the gRNA is single-stranded guide RNA (sgRNA), and the first gRNA preferably binds upstream of the donor sequence on the sense strand, the second gRNA preferably binds downstream of the donor sequence on the anti-sense strand, and the third gRNA preferably binds the target locus on the anti-sense strand.

In various embodiments, the systems are for integrating a detectable marker into a target locus (e.g., an intron of a target gene), wherein the detectable marker is provided in a FlipTrap cassette, and the FlipTrap cassette is in a donor sequence.

Some embodiments provide that a donor sequence (e.g., containing a FlipTrap cassette) in provided in a nucleic acid vector comprising a nucleic acid backbone sequence, a splice acceptor sequence, a splice donor sequence, a first recombination site and a second recombination site forming a first recombination site pair capable of being recognized by a first recombinase, a third recombination site and a fourth recombination site forming a second recombination site pair capable of being recognized by the first recombinase or a second recombinase, and a polyadenylation sequence, wherein the first and second recombination sites are in opposite orientations, wherein the third and fourth recombination sites are in opposite orientations, and wherein the second recombination site pair flanks the second recombination site, but does not flank the first recombination site. Further embodiments provide the system also includes one or more recombinases, such that when introduced into the target cell, the one or more recombinases create a first recombination and optionally a second recombination event or create a second recombination event depending on the introduced recombinase.

Various embodiments provide methods for integrating a donor sequence in a target locus or a locus of interest in a target cell, wherein the donor sequence provided with the three gRNA system and a quantity of a Cas nuclease (e.g., Cas9 nuclease) results in greater integration efficacy/efficiency, compared with a donor sequence provided with only a gRNA targeting the target locus (and with or without a Cas nuclease), or compared with a donor sequence provided with two flanking gRNA (and with or without a Cas nuclease), or compared with a donor sequence without any flanking gRNA or a gRNA targeting the target locus (and with or without a Cas nuclease).

Further embodiments provide that the methods include pre-mixing the donor sequence with the first and second gRNA in the presence of a quantity of a Cas nuclease (e.g., Cas9 nuclease) to form DNA:RNP complexes, separately pre-mixing the third gRNA and a quantity of a Cas nuclease to form another DNA:RNP complex, and introducing the complexes to a target cell, e.g., via microinjection, to induce gene editing.

### BRIEF DESCRIPTION OF THE FIGURES

FIGS. 1 and 2 depict an exemplary FlipTrap vector in a gene trapping approach. The exemplary FlipTrap cassette forms a full-length functional fusion protein with a green fluorescent tag (citrine, YFP variant) when inserted into an intron. When exposed to Cre recombinase, the cassette assumes a second conformation, and generates a red fluorescent tag (mCherry) gene trap and a mutant allele for the trapped gene. Thus, each flip trap line can be used to reveal the protein expression pattern (using the citrine fusion trap) and the mutant phenotype (using the mCherry gene trap). FIG. 1 depicts a transposon-based gene-trapping technology called ‘flip trapping’. The Tol2 element is an autonomously active transposon, containing a gene encoding a complete and functional transposase that is capable of identifying, excising, and reinserting the DNA element defined by its inverted terminal repeats (ITR) or other elements with the same ITRs. FIG. 2 depicts that the exemplary FlipTrap vector consists essentially of a citrine coding sequence flanked by intronic sequences that contain a splice acceptor (SA) and a splice donor (SD). In the reverse orientation are mCherry and polyA sequences. Heterotypic lox sites are shown for loxP and loxPV. FRT sites are positioned internal to the transposable elements (TEs). FIG. 2 also depicts that insertion of the exemplary FlipTrap by transposition into the intron of an actively expressed gene leads to splicing of the citrine exon, allowing the formation of an endogenously expressed full-length fluorescent fusion protein when the insertion is in-frame with the trapped gene.

FIG. 3 depicts an exemplary use of a trio sgRNA system in a gene trapping approach with a FlipTrap vector exemplified in FIGS. 1 and 2. Here, the trio sgRNA system includes a sgRNA that targets the 5′ end upstream of the donor DNA (“sgRNA2-5′ donor”), a sgRNA that targets the 3′ end downstream of the donor DNA (“sgRNA3-3′ donor”), and a sgRNA that targets a locus of interest in a target cell (“sgRNA1-target”).

FIG. 4 depicts a recreation of FlipTrap insertion into hmga2 locus of zebra fish.

FIGS. 5A and 5B depict synergy of trio sgRNA for donor DNA integration. FIG. 5A depicts a diagram showing visualization of a system containing three sgRNAs (a trio sgRNA system), a donor plasmid containing FlipTrap gene, and a target/host cell's locus of interest (e.g., hmga2 locus), wherein (1) a sgRNA targets the 5′ end upstream of the donor DNA's (“donor sgRNA2-5′”; it binds the sense strand, so targeting—being homologous to—the anti-sense strand), (2) a sgRNA targets the 3′ end downstream of the donor DNA (“donor sgRNA3-3′”; it binds the anti-sense strand, so targeting—being homologous to—the sense strand), and (3) a sgRNA targets an intron of the hmga2 locus (“hmga2 sgRNA1”; it binds the anti-sense strand, so targeting—being homologous to—the send strand). FIG. 5B depicts a graph showing that the trio sgRNA system results in integration of the FlipTrap gene from a plasmid donor DNA (“donor DNA+3sg RNA”) to an intron of hmga2 locus, therefore generating fusion protein of HMGA2 tagged with citrine, at a much higher percentage, compared to the plasmid donor DNA with only 2 sgRNAs, or compared to cut/linearized DNA with only 1 sgRNA that targets host cell's hmga2 intron locus.

FIG. 5C shows wide-field fluorescent image of positive donor DNA integration as determined by Citrine expression (in the head region and trunk of embryo at 32hpf) and confocal fluorescent image of positive somatic integration at hmga2 locus based on Citrine fusion protein expression in the nucleus (green), expression of mCherry (red) and the vital stain FM4-64fx (blue). Scale bar=50 umicron.

FIG. 6A-6C depict NHEJ-mediated integration of donor DNA. FIG. 6A is a schematic of a FlipTrap donor DNA and a hmga2 target locus, showing the sequences for the donor sgRNA binding sites and for the target sgRNA binding site. Green and red rectangle represent coding sequencing for the citrine and mCherry exons, respectively, in the FlipTrap construct. PAM sequence in yellow, 5′ donor sgRNA binding sequence in blue, 3′ donor sgRNA sequence in purple. The nucleotide sequence shown in FIG. 6A upstream of the green and red rectangle in the donor DNA is presented in the 5 to 3 direction by SEQ ID NO:19. The nucleotide sequence shown in FIG. 6A downstream of the green and red rectangle is presented in the 5 to 3 direction by SEQ ID NO:20. The nucleotide sequence shown in FIG. 6A of target locus hmga2 is presented in the 5 to 3 direction by SEQ ID NO:21. FIG. 6B depicts sequencing from embryos injected with preassembled DNA:RNP complexes containing FlipTrap plasmid, 5′ and 3′ donor and targeting sgRNAs and Cas9 protein. DNA:RNP complexes containing all plasmid DNA and all three sgRNA integrates with indels and imprecise excision of the donor DNA. While the main vector sequence (depicted as green and red rectangles in FIG. 6B) that is within the donor sgRNA binding is intact and does not show aberrant recombination, we have aberrant recombination of the linear DNA/relevant gene trapping cassette when no donor sgRNA with Cas9 (RNP) are used (see FIG. 6C). FIG. 6C depicts sequencing from embryos injected with preassembled DNA:RNP complexes containing linear FlipTrap DNA, targeting hmga2 sgRNA and Cas9 protein. DNA:RNP complexes containing linear DNA and only targeting sgRNA leads to aberrant recombination with inversions and deletions of the donor DNA. Color code for sequences in 16B and 16C as follows: black sequence from hmga2 locus, blue sequence from 5′ end of the donor DNA, purple sequence from 3′ end of donor DNA, and red sequence represent indels. Dots represent sequences not shown. Green and red rectangle represent coding sequencing for citrine and mCherry, respectively, in the donor construct.

FIG. 7 depicts the increased survival with linear donor DNA and that with either linear donor DNA or plasmid donor DNA, a trio sgRNA system results in a higher percentage gene editing (characterized by YFP positive percentage) than a sgRNA alone which targets host cell (target sgRNA) or in combination with one sgRNA that targets 5′ or 3′ donor DNA.

FIG. 8 depicts synergy of trio sgRNA with dCas9 and linear DNA. The diagram shows, an exemplary trio sgRNA system, which has two pre-formed ribonucleoprotein (RNP) complexes: one complexing among the 5′ end of the linear donor DNA, a sgRNA that targets the 5′ end of the linear donor DNA (“5′ donor sgRNA”), and a nuclease-deactivated Cas9 (dCas9), and the other complexing among the 3′ linear donor DNA, a sgRNA that targets the 3′ end of the donor DNA (“3′ donor sgRNA”), and a dCas9. The graph shows that with linear donor DNA, a trio sgRNA system (including pre-formed RNPs containing dCas9 for linear donor DNA) results in a much higher somatic integration than a sgRNA alone which targets host cell (target sgRNA) or in combination with one sgRNA that targets 5′ donor DNA.

FIG. 9A-9C depict that directionality of sgRNA affects donor DNA integration efficiency. FIG. 9A is a schematic denoting DNA strand that donor sgRNAs (orange and green) and target sgRNAs (purple) bind to for the FlipTrap donor DNA and hmga2 target locus, respectively. 5′ donor sgRNA1 is also denoted as “FT5-1” in Example 10; 5′ donor sgRNA2 is denoted as “FT5-2”; 3′ donor sgRNA2 is denoted as “FT3-2”; 3′ donor sgRNA1 is denoted as “FT3-1”; hmga2 sgRNA1 is denoted as “hmga2-1”; and hmga2 sgRNA2 is denoted as “hmga2-2”. FIG. 9B is a barplot of donor DNA integration efficiency as determined by citrine expression at the hmga2 locus with DNA:RNP complex injections using combinations of donor sgRNAs and target sgRNAs denoted in 9A. Highest efficiency observed with 5′ donor sgRNA binding the sense strand and target sgRNA binding the anti-sense strand. N=number of independent injections experiments, n=total number of embryos injected. FIG. 9C depicts HRMA temperature-shifted curves showing the peak melting profile of PCR amplicons from single embryos injected with the hmga2-sgRNA2 and Cas9 protein RNP complex (embryos1-6) or wildtype controls. Asterisk (*) denotes individual embryos with shift in melting profile as compared to wildtype controls, indicating CRISPR/Cas9 induce indels. Relative Fluorescence Units (RFU).

FIG. 10 depicts that Cas9 storage buffer affects efficiency. Therefore, an exemplary storage buffer contains 20 mM HEPES (4-(2-hydroxyethyl)-1-piperazineethanesulfonic acid), 150 mM potassium chloride (KCl), and 1% sucrose at pH 7.5.

FIG. 11 depicts that single site DNA cleavage is sufficient for DNA integration in a trio sgRNA system, each sgRNA coupled with a nicking enzyme, nCas9, which retains the modular RuvC-like domain and can cleave one of the DNA strands.

FIG. 12 depicts that assymetric DNA donor can enhance homology-directed repair (HDR) in genome editing. Before complete dissociation of Cas9 from double-stranded DNS substrates, Cas9 asymmetrically releases the 3′ end of the cleaved DNA strand that is not complementary to the sgRNA (non-target strand). That is, the non-target DNA strand (i.e., strand not targeted by the sgRNA) is released before the target strand (i.e., strand targeted by the sgRNA). Therefore designing single-stranded DNA (ssDNA) donors complementary to the strand that is released first, i.e., the non-target strand, increases the rate of HDR in cells (˜10%) using Cas9 or nickase variants. For example, HDR was observed with ssDNA and catalytically inactive Cas9 mutant (dCas9) in Richardson, et al., Nature Biotechnology, 34, 339-344 (2016).

FIG. 13 depicts a summary of design approach. Trio sgRNA strategy enhances integration efficiency of donor DNA. Trio sgRNA synergize to increase efficiency. Synergy is independent of active Cas9. It is possible that there is potential interaction between Cas9RNP complexes. When integration of donor DNA is mediated by NHEJ repair, Cas9-RNP complex is protective of aberrant integration. Efficiency of DNA integration is dependent on directionality of sgRNA, e.g., guide RNAs targeting complementary strand for donor and for target DNA affect the integration as shown in FIG. 9.

FIGS. 14A and 14B depict trio sgRNA CRISPR/Cas system and cutting efficiency of individual sgRNA. FIG. 14A is a schematic of trio sgRNA strategy for integration of a gene trap construct into the hmga2 locus. The gene trap construct, FlipTrap, consists of citrine coding sequence (green) flanked by intronic sequences that contain a splice acceptor (SA, beige) and donor (SD, beige). In the reverse orientation are sequences encoding mCherry (red) and polyA (red). Heterotypic lox sites are shown for loxP (light grey) and loxPV (dark grey). Donor sgRNA directs integration of donor plasmid into target locus while two donor sgRNA, 5′ and 3′ of desired integration sequence, allows cleavage and prevents integration of plasmid backbone. FIG. 14B shows DNA agarose gels for in vitro sgRNA cutting efficiency test of target sgRNAs and of donor sgRNAs. (−) denotes samples without sgRNA while (+) denotes samples incubated with the respective sgRNAs.

FIGS. 15A and 15B depict efficient DNA integration in the absence of donor DNA cleavage. FIG. 15A depict a schematic of preassembled DNA:RNP complex used in linear donor DNA experiments. DNA:RNP complexes were preassembled containing linear DNA and donor sgRNAs with either Cas9or dCas9, whereas separately, targeting RNP complexes were assembled containing only target sgRNA (sgRNA that binds target locus of interest) and Cas9. The DNA:RNP complexes were then mixed with the targeting RNP complex prior to embryo microinject. FIG. 15B is a bar plot showing integration efficiency of preassembled DNA:RNP complexes containing linear DNA and Cas9 RNP complex or dCas9 RNP complex as illustrated in 15A and combination of donor and target sgRNAs. Results are shown as the averages±standard error of the mean from “+” indicates the N=number of independent injections experiments, n=total number of embryos injected. Asterisk denotes experimental conditions with no statistical differences using Student's t test (*p=0.38)

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22nd ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3rd ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Indeed, the present invention is in no way limited to the methods described herein. For purposes of the present invention, the following terms are defined below.

The term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

“Administering” and/or “administer” as used herein refer to any route for delivering a pharmaceutical composition to a patient. Routes of delivery may include non-invasive peroral (through the mouth), topical (skin), transmucosal (nasal, buccal/sublingual, vaginal, ocular and rectal) and inhalation routes, as well as parenteral routes, and other methods known in the art. Parenteral refers to a route of delivery that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

“Modulation” or “modulates” or “modulating” as used herein refers to upregulation (i.e., activation or stimulation), down regulation (i.e., inhibition or suppression) of a response or the two in combination or apart.

“Pharmaceutically acceptable carriers” as used herein refer to conventional pharmaceutically acceptable carriers useful in this invention.

“Promote” and/or “promoting” as used herein refer to an augmentation in a particular behavior of a cell or organism.

“Subject” as used herein includes all animals, including mammals and other animals, including, but not limited to, companion animals, farm animals and zoo animals. The term “animal” can include any living multi-cellular vertebrate organisms, a category that includes, for example, a mammal, a bird, a simian, a dog, a cat, a horse, a cow, a rodent, and the like. Likewise, the term “mammal” includes both human and non-human mammals.

“Therapeutically effective amount” as used herein refers to the quantity of a specified composition, or active agent in the composition, sufficient to achieve a desired effect in a subject being treated. A therapeutically effective amount may vary depending upon a variety of factors, including but not limited to the physiological condition of the subject (including age, sex, disease type and stage, general physical condition, responsiveness to a given dosage, desired clinical effect) and the route of administration. One skilled in the clinical and pharmacological arts will be able to determine a therapeutically effective amount through routine experimentation.

“Treat,” “treating” and “treatment” as used herein refer to both therapeutic treatment and prophylactic or preventative measures, wherein the object is to prevent or slow down (lessen) the targeted condition, disease or disorder (collectively “ailment”) even if the treatment is ultimately unsuccessful. Those in need of treatment may include those already with the ailment as well as those prone to have the ailment or those in whom the ailment is to be prevented.

Generally, Cas9 forms a nucleoprotein complex with a single guide RNA (sgRNA) containing a 20 nt sequence that determines binding specificity based on Watson-Crick base pairing. With the commonly used Cas9 protein from Streptococcus pyogenes (SpCas9), the only sequence requirement in the genomic target is an NGG (or, less optimally, an NAG) PAM motif (where N signifies any nucleotide) directly downstream from the binding sequence.

Nuclease-deactivated Cas9, or Cas9 endonuclease dead, also known as dead Cas9 or dCas9, is a mutant form of Cas9 whose endonuclease activity is removed through point mutations in its endonuclease domains. Features of dCas9 includes, but are not limited to, dCas9-mediated transcription regulation and visualization of DNA sequences in living cells.

A nicking enzyme mutant of Cas9, also known as nCas9, cleaves one of the DNA strands. Cas9 contains two catalytic domains, the modular RuvC-like domain and the C-terminal HNH-like domain. Each domain cleaves one of the DNA strands, resulting in a blunt-ended DSB or short overhang 3 bp upstream of the PAM motif. Mutation of the active site in either catalytic domain turns wild-type Cas9 (Cas9WT) into a nicking enzyme (nCas9), while mutating both active sites renders it catalytically dead (dCas9), but still able to efficiently bind DNA. For example, the Cas9^{D10A} variant with a mutation in the active site of the RuvC-like domain cleaves the DNA strand complementary to the sgRNA-binding sequence, while Cas9^{H840A} with a mutation in the HNH-like domain cleaves the noncomplementary strand, and cas9^{D10A/H840A} is catalytically dead.

Guide RNA (gRNA) is a piece of RNAs that function as guides for RNA- or DNA-targeting enzymes, which they form complexes with. For example, for a DNA-targeting enzyme, Cas9, the single-stranded guided RNA (sgRNA) makes a complex with the Cas9 protein to cleave the DNA. Single guide RNA (sgRNA) contains a targeting sequence (crRNA sequence) and a Cas9 nuclease-recruiting sequence (tracrRNA). Generally, the crRNA region is a 20-nucleotide sequence that is homologous to a region in the gene of interest and will direct Cas9 nuclease activity. For example, if the targeting sequence (crRNA region) of a sgRNA targets (therefore being homologous to) a region of the sense strand in a gene of interest, then the targeting sequence of the sgRNA binds (therefore being complementary to) the anti-sense strand in the corresponding region of the gene of interest. In other instances, the crRNA region is a sequence of 15-25 nucleotides long. For example, the crRNA region is a 19-nucleotide sequence; the crRNA region is an 18-nucleotide sequence; the crRNA region is a 17-nucleotide sequence; the crRNA region is a 21-nucleotide sequence; the crRNA region is a 22-nucleotide sequence; or the crRNA region is a 23-nucleotide sequence.

Upstream is toward the 5′ end of the nucleic acid molecule and downstream is toward the 3′ end. When considering double-stranded DNA, upstream is toward the 5′ end of the coding strand (sense strand) for the gene in question and downstream is toward the 3′ end. Due to the anti-parallel nature of DNA, this means the 3′ end of the template strand (anti-sense strand) is upstream of the gene and the 5′ end of the template strand (anti-sense strand) is downstream.

The Inventors have generated a multi-functional gene trap that forms fluorescent fusion proteins to either full-length or truncated proteins at a trapped locus. The gene trap vector, termed FlipTrap, incorporates an internal exon encoding the fluorescent protein citrine as an example, into the genome by Tol2 transposable elements. The Inventors had implemented this gene trapping approach in zebrafish using random insertion into the genome by Tol2 transposition. While the approach has identified >200 gene trap lines that are a powerful set of reagents to perform quantitative analysis of protein expression, localization and dynamics in a vertebrate, the randomness of Tol2 transposition prevents targeted tagging of select protein of interest. However, the FlipTrap vector functions when integrated within introns, precluding the need for precise HDR upon integration to tag proteins. Therefore, the Inventors coupled the FlipTrap approach with the targeting capabilities of the CRISPR-Cas system to achieve protein tagging with high efficiency. Here, the Inventors demonstrate the efficiency of the trio sgRNA approach.

Various embodiments provide a system comprising multiple gRNA, for integrating a donor sequence, or in some embodiments a donor sequence comprising a FlipTrap cassette, into a target locus of interest. In various embodiments, a system comprises at least three gRNA with one or more RNA-guided nucleases for integrating a donor sequence, or in some instances a donor sequence comprising a FlipTrap cassette, into a target locus of interest. In some embodiments, a system comprises a donor sequence with flanking gRNA:Cas nuclease complexes (one complex positioned upstream of the donor sequence, and another complex positioned downstream of the donor sequence), and a gRNA targeting a locus of interest (e.g., the locus of interest is in a genomic DNA of the host cell), as well as another Cas nuclease that is recruited to the gRNA targeting the locus of interest which can be supplied to form a complex with the gRNA targeting the locus of interest.

In some embodiments, a system comprises, consists essentially of, or consists of three gRNA, wherein the first and second gRNA flank a donor sequence, and the third gRNA binds a target locus of interest.

In some instances, the first gRNA contains a sequence homologous to, or complementary with, a segment upstream of the donor sequence; and the second gRNA contains a sequence homologous to, or complementary with, a segment downstream of the donor sequence; thereby the first gRNA and the second gRNA flank the donor sequence.

In some instances, the first gRNA and the second gRNA independently bind the sense strand or the anti-sense strand, as long as the binding by the first gRNA and the binding by the second gRNA are one upstream and one downstream of the donor sequence, i.e., the donor sequence is internal to the binding sites by the first gRNA and the second gRNA.

In further instances, the donor sequence has at least one binding site for gRNA, or two or more binding sites for two or more gRNA, upstream of the donor sequence, and it has at least one binding site for a gRNA, or two or more binding sites for two or more gRNA, downstream of the donor sequence.

In various instances, the binding of substrate DNA by a gRNA positioned upstream of a donor sequence, downstream of a donor sequence, and/or at a locus of interest (e.g., in genomic DNA) further implies that an RNA-guided nuclease (e.g., Cas9) is recruited at the binding position.

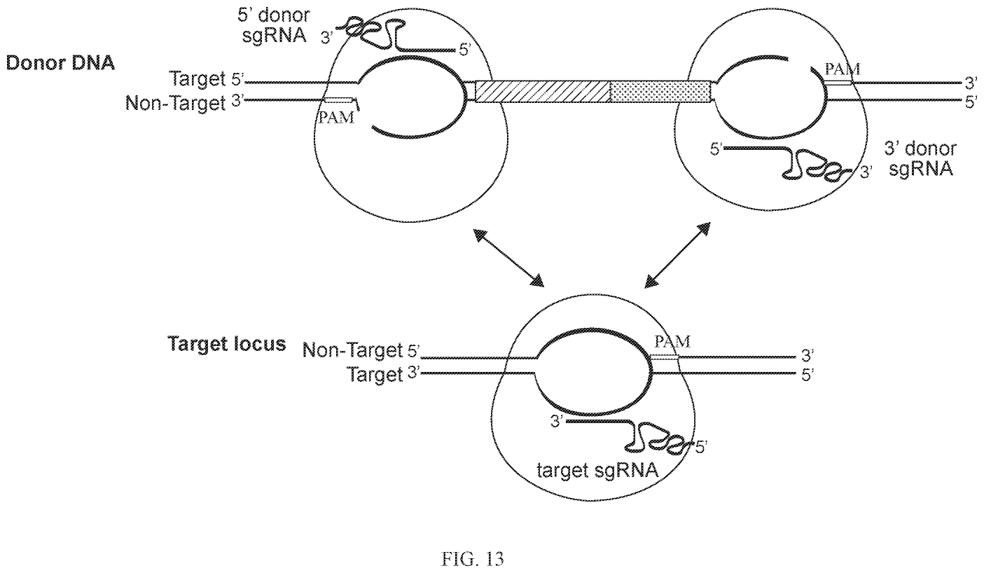

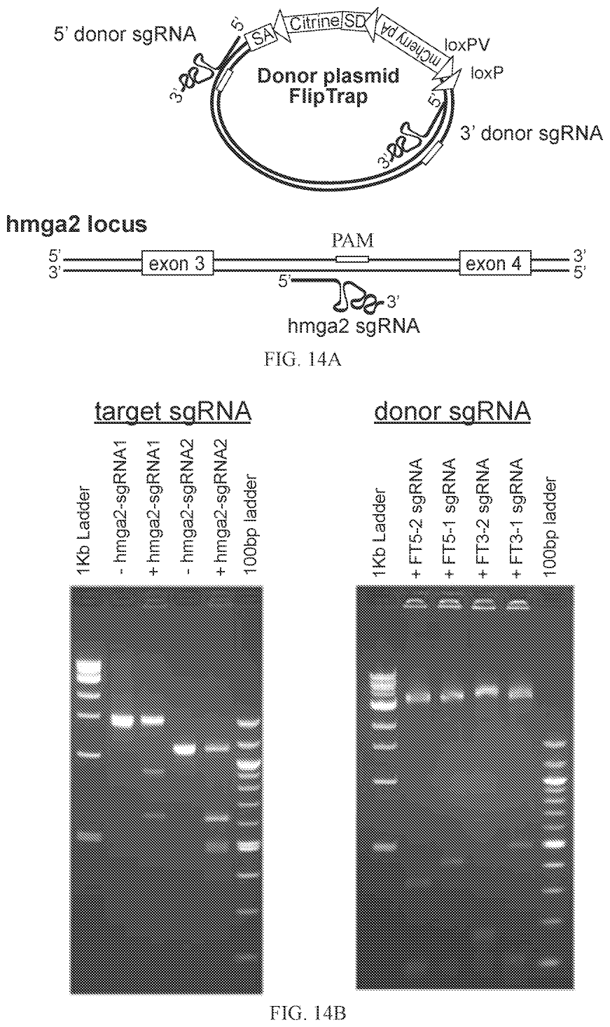

In various instances directionality of a sgRNA in targeting a single strand of the target locus (e.g., host cell DNA) or of the donor DNA in provided. For example, at least one sgRNA targets the 5′ end of the sense strand of the donor DNA, which is annotated as “5′ donor sgRNA”, and at least one sgRNA targets the anti-sense strand of the target locus (e.g., host cell DNA), which is annotated as “target sgRNA”. See FIG. 13.

In some embodiments, a first sgRNA of the multi- (or trio) sgRNA system binds (or contains a sequence complementary with) the 5′ end of the sense strand (coding strand) for a donor sequence. Or that is, it targets (or contains a sequence homologous to) the 3′ end of the anti-sense strand for the donor sequence.

In some embodiments, a second sgRNA of the multi- (or trio) sgRNA system binds (or contains a sequence complementary with) the 5′ end of the anti-sense strand for a donor sequence. Or that is, it targets (or containing a sequence homologous to) the 3′ end of the sense-strand for the donor sequence.

In some embodiments, a third sgRNA of the multi- (or trio) sgRNA system binds the anti-sense strand of a target locus of interest, or a locus of interest in a target cell (e.g., a host cell intended for gene editing, where the donor sequence is intended to integrate to). That it, at least one sgRNA targets (therefore containing a sequence homologous to) a segment of the sense strand of the locus of interest in the target cell.

In some embodiments, the multi-gRNA system (e.g., a trio sgRNA system) includes at least one sgRNA that binds the 5′ end of the sense strand for a donor sequence (upstream of the donor sequence), at least one sgRNA that binds the anti-sense strand of a locus of interest in a target cell, and at least one sgRNA that binds a segment downstream of the donor sequence. In some aspects, the at least one sgRNA that binds a segment downstream of the donor sequence binds the 5′ end of the anti-sense strand for the donor sequence; and in other aspects, the at least one sgRNA that binds a segment downstream of the donor sequence binds the 3′ end of the sense strand for the donor sequence.

In some embodiments, the multi-gRNA system is a trio sgRNA system, which includes at least one sgRNA that binds the 5′ end of the sense strand (coding strand) for a donor sequence, at least one sgRNA that binds the 5′ end of the anti-sense strand (template strand) for the donor sequence, and at least one sgRNA that binds the anti-sense strand of a locus of interest in a target cell.

In other words, in some embodiments, the multi-gRNA system is a trio sgRNA system, which includes at least one sgRNA that targets (e.g., contains a crRNA region that is homologous to) the 3′ end of the anti-sense strand for the donor sequence (upstream of the donor sequence), at least one sgRNA that targets (e.g., contains a crRNA region that is homologous to) the 3′ end of the sense-strand for the donor sequence (downstream of the donor sequence), and at least one sgRNA that targets (e.g., contains a crRNA region that is homologous to) the sense strand of a locus of interest in a target cell.

A genomic DNA region that corresponds to the crRNA sequence of the sgRNA can be selected based on the following procedures. The 3′ end of the genomic DNA sequence targeted by (homologous to) the crRNA sequence has a proto-spacer adjacent motif (PAM) sequence (5′-NGG-3′), but the PAM sequence is not included in the design of the sgRNA sequence. The 20 nucleotides (or 15-25 nucleotides) upstream of the PAM sequence in the genomic DNA will be the targeting sequence (crRNA), and the RNA guided nuclease (e.g., Cas9 nuclease) will cleave approximately three bases upstream of the PAM. The target sequence can be on either DNA strand, and the PAM sequence is on the 3′ end of the target sequence. Online tools (e.g., CRISPR Design or CHOPCHOP) can detect PAM sequences and list possible crRNA sequences within a specific DNA region, and these algorithms also predict off-target effects elsewhere in the genome.

Once sgRNA sequences is designed, sgRNA can be synthesized (e.g., with Guide-it sgRNA In Vitro Transcription Kit). For example, polymerase chain reaction (PCR) is used to generate a template DNA containing the sgRNA-encoding sequence (constituted by the crRNA of design and a tracRNA) under the control of a T7 promoter. Then, in vitro transcription using the PCR product template is used to produce sgRNAs that can be purified for efficiency testing and/or cell transduction.

Various embodiments provide that a system for integrating one or more donor sequences into one or more target loci of interest, wherein the system comprises (1) the one or more donor sequences, (2) a pair of two gRNA for each of the one or more donor sequences, wherein the two gRNA in a pair flank the respective donor sequence (e.g., the two gRNA in a pair bind upstream and downstream of the respective donor sequence), (3) a gRNA for each of the one or more target loci of interest.

Further embodiments provide that the system for integrating one or more donor sequences into one or more target loci of interest further comprises (4) a quantity of RNA-guided nuclease. In some aspects, the RNA-guided nuclease is Cas9, including wild-type Cas9, a nicking enzyme variant of Cas9 (nCas9) or SpCas9 nickase (Cas9n), or deactivated Cas9 (dCas9) or nuclease-dead SpCas9. In some aspects, the RNA-guided nuclease comprises Cas9 (wild type Cas9, or Cas9 capable of cleaving both strands of DNA). In other aspects, the RNA-guided nuclease comprises Cas12a (formerly Cpf1), Cas12b, and CasX (also known as Cas12e). In further aspects, the RNA-guided nuclease is Cas 9 from S. pyogenes (SpCas9). In further aspects, the RNA-guided nuclease is Cas 9 from S. aureus (SaCas9) or Cas9 from C. jejuni (CjCas9). In further aspects, the RNA-guided nuclease is an engineered nuclease, such as a high-fidelity SpCas9 (SpCas9-HF1, eSpCas9-1.1) and SpCas9 with relaxed PAM (SpCas9-NG). In further aspects, the RNA-guided nuclease is a Cas nuclease selected from SpCas9, NmCas9, SaCas9, FnCas9, St1Cas9/St3Cas9, CjCas9, Cas9n, dCas9, SpCas9-HF1, eSpCas9-1.1, SpCas9-NG, Cas12a, Cas12b, and Cas12e.

Yet further embodiments provide that in the system for integrating one or more donor sequences into one or more target loci of interest, each of the two gRNA in the pair together with a Cas9 nuclease and a segment upstream or downstream of the donor sequence form a complex (DNA:RNA:nuclease complex), and the gRNA for a target loci of interest together with a Cas9 nuclease form a complex (RNA:nuclease complex).

Donor sequences can be any sequence of choice to be integrated into a target locus or to a target cell. In various embodiments, a donor sequence is provided in a nucleic acid vector. In some aspects, the nucleic acid vector containing the donor sequence is a plasmid. In other aspects, the donor sequence is a linearized DNA, or linear DNA without a vector backbone.

In some embodiments, the donor sequence encodes a detectable marker, such as a visible marker or a selectable marker. In some embodiments, the donor sequence comprises a detectable marker sequence, configured for integration into an intron of a target cell, and therefore the donor sequence further comprises a splice acceptor sequence and a splice donor sequence. In some embodiments, the donor sequence is a FlipTrap cassette (or a FlipTrap vector), for generating genetic alleles that in the initial conformation make a fusion protein with a given marker (e.g., a first detectable marker) and after conditional recombination create a mutant allele with a second marker (e.g., a second detectable marker). In further embodiments, a FlipTrap cassette is integrated into the genome of a target cell site specifically via the multi- (or trio) gRNA system provided herein; rather than to be integrated randomly.

In various embodiments, a FlipTrap cassette can generally be provided in a nucleic acid vector that comprises (1) a nucleic acid backbone sequence, (2) a splice acceptor sequence, (3) a splice donor sequence, (4) a first recombination site and a second recombination site forming a first recombination site pair capable of being recognized by a first recombinase, wherein the first and second recombination sites are in opposite orientations, (5) a third recombination site and a fourth recombination site forming a second recombination site pair capable of being recognized by the first recombinase or a second recombinase, wherein the third and fourth recombination sites are in opposite orientations, and wherein the second recombination site pair flanks the second recombination site, but does not flank the first recombination site, and (6) a polyadenylation sequence.

In some instances, the second pair of recombination sites and the first pair are different sequences. In some instances, the recombination sites can be recognized by the same recombinase or by different recombinases. In some instances, the second pair of recombination sites flank a second marker protein that has a polyadenylation site. In some instances, the splice donor sequence is flanked by the first pair of recombination sites, but not by the second pair of recombination sites.

In some embodiments, a FlipTrap cassette comprises (1) a nucleic acid backbone sequence, (2) a splice acceptor sequence, (3) a splice donor sequence, and (4) a detectable marker sequence.

If a FlipTrap cassette integrates into an intron in the same orientation as the endogenous gene, then splicing will occur between the upstream endogenous exon and the cassette's splice acceptor (SA) and between the cassette's splice donor (SD) and the downstream endogenous exon generating a transcript encoding a fusion protein. Such an allele is called a “Fusion Trap” because it traps the splicing signals of an endogenous gene to generate a fusion protein if the construct is in the correct orientation and in-frame.

Upon addition of the site-specific recombinase(s) that recognize the two sets of recombination sites, recombination will occur such that the first detectable marker and the SD will be deleted and the second detectable marker and the poly-A (pA) sequence will now be in the sense orientation with respect to the endogenous gene. This conformation is called a “Gene Trap” because it traps the upstream exon, but then terminates the transcript prematurely because of the pA sequence.

A cell or organism is put in contact or introduced with (e.g. transformed) with a vector, such that the vector integrates into the cell's DNA. A cell or organism put in contact with a vector of the present disclosure is the receiving cell or receiving organism, respectively. In one embodiment, the vector integrates into the cell's genomic DNA. In some embodiments, the receiving cell or receiving organism is able to express one or more recombinases in order to induce recombination of the first and second pairs of recombination sites.

In some embodiments, any marker can be used in a donor sequence or more specifically in a FlipTrap cassette. In one embodiment, a visible marker is a fluorescent protein, such as green fluorescent protein, yellow fluorescent protein, red fluorescent protein, citrine, and mCherry. In one embodiment, a visible marker is an enzyme that can be used with chromogenic substrates, e.g., beta-galactosidase. In one embodiment, a selectable marker is an antibiotic resistance gene, e.g., neomycin resistance gene. In various embodiments, the first detectable marker and the second detectable marker in the FlipTrap cassette can independently be or comprise a visible maker, a selectable marker, or both.

In further embodiments, sequences encoding the first detectable marker and/or the second detectable marker is at least 200 bp in length, but no more than 1500 bp. In some instances, the sequence encoding a detectable marker is between 500-1000 bp in length for inclusion in a FlipTrap cassette. In some instances, the sequence encoding a detectable marker is 300-400 bp in length for inclusion in the donor sequence. In some instances, the sequence encoding a detectable marker is 400-500 bp in length for inclusion in the donor sequence. In some instances, the sequence encoding a detectable marker is 500-600 bp in length for inclusion in the donor sequence. In some instances, the sequence encoding a detectable marker is 600-700 bp in length for inclusion in the donor sequence. In some instances, the sequence encoding a detectable marker is 700-800 bp in length for inclusion in the donor sequence. In some instances, the sequence encoding a detectable marker is 800-900 bp in length for inclusion in the donor sequence. In some instances, the sequence encoding a detectable marker is 900-1000 bp in length for inclusion in the donor sequence.

In various embodiments, a donor sequence for use in a multi- (trio-) gRNA system or in a genomic editing method disclosed herein, can be a large polynucleotide, such as at least 200 bp in length or at least 200 nt in length. In some instances, a donor sequence is at least 300 bp (or 300 nt when provided as a single strand), at least 400 bp (or 400 nt when provided as a single strand), or at least 500 bp, 600 bp, 700 bp, 800 bp, 900 bp, 1000 bp, 1300 bp, 1500 bp, 1700 bp, or at least 2000 bp in length; or up to 5000 bp in length. The large polynucleotide can be efficiently incorporated in target loci of interest with the multi- (or trio-) gRNA systems or methods disclosed herein, resulting in to at least 20%, 25%, 30%, 31%, 32%, 33%, 34%, 35%, 36%, 37%, 38%, 39%, 40%, 41%, 42%, 43%, 44%, 45%, or 50%; and in some instances preferably at least 40% of a large donor sequence (at least 200 bp, at least 500 bp, or at least 1000 bp) is detected or incorporated into the genomic DNA of a target cell.

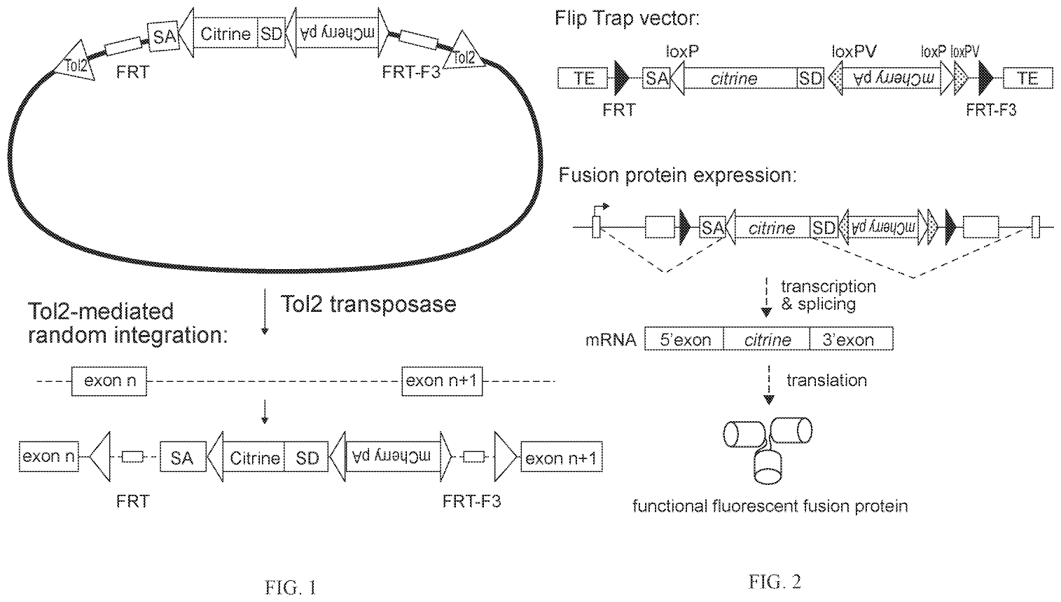

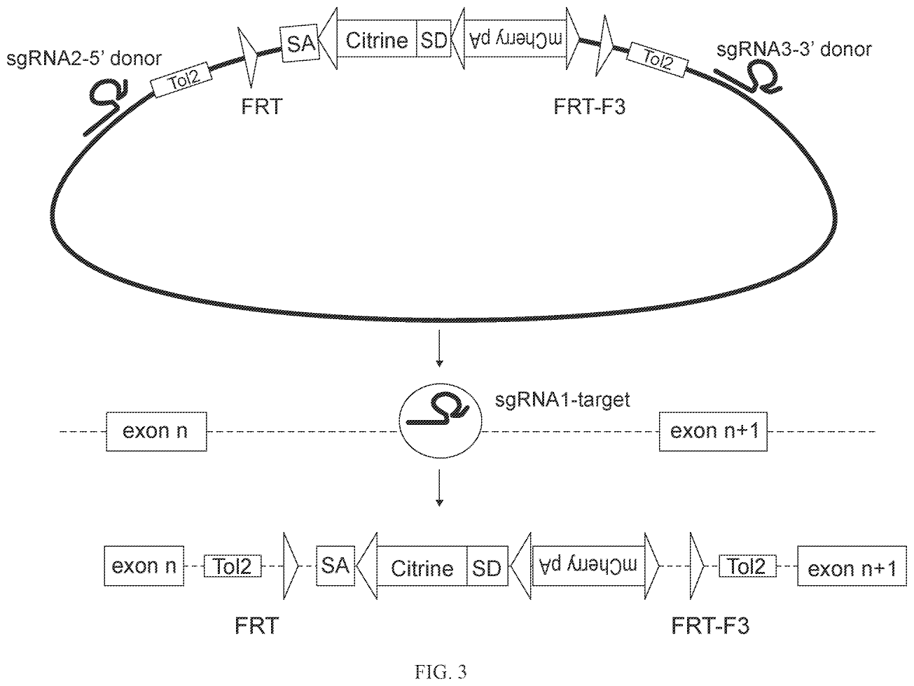

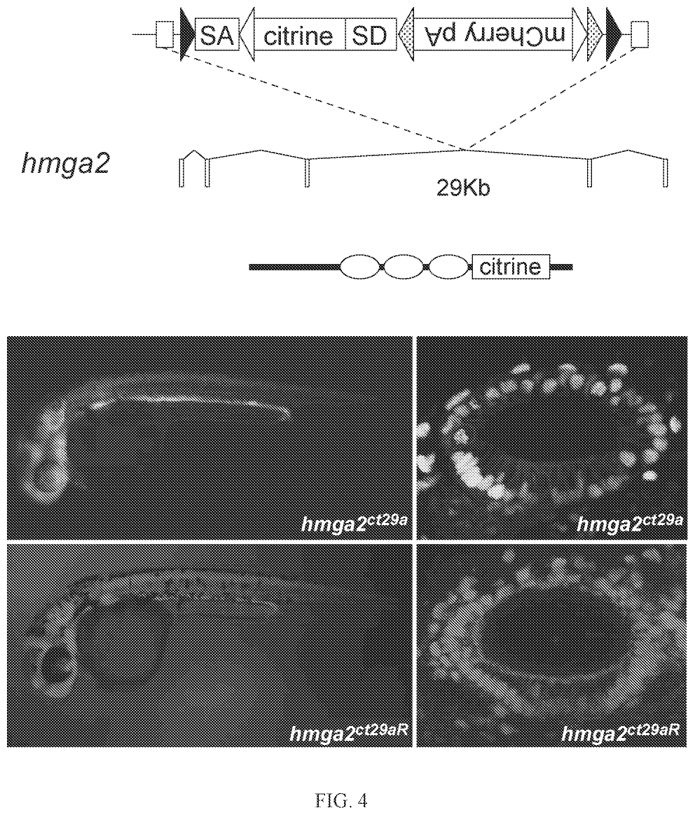

In various embodiments, any enzyme that causes recombination between two sites based on a specific sequence can be used for the recombinases in the FlipTrap vector. In some embodiment, the recombinases used in the FlipTrap vector of the present disclosure include, but are not limited to the Cre and FLP recombinases. In one embodiment, two different recombinases can be used along with their respective two cognate recombination sites. In another embodiment a single recombinase is used for two different pairs of heterotypic sites. For example, the Cre recombinase can be used along with one pair of loxP sites and one pair of loxPV sites. In various embodiments, the relative order and orientation of the recombination sites are as indicated in FIG. 2 or 1.

In some embodiments, a transposon-based vector is used to insert the FlipTrap cassette, such as Tol2, and Sleeping Beauty (U.S. Pat. No. 6,613,752, which is hereby incorporated by reference). Since transposon-based vectors require minimal extraneous sequence to be added to the vector, these can work with high efficiency, and can target a very large number of sites in the genome.

In some embodiments, the sequences for SA and SD, are those of the splice acceptor and donor from a naturally occurring large internal exon from the same species as the receiving cell or organism. Such large internal exons can be identified through sequence analysis in organisms with a substantial genomic sequence database. In one embodiment, the SA sequence includes about 300 bp upstream of the intron-exon boundary and about 15 bp downstream of the intron-exon boundary. These flanking sequences added to the SA sequence can help ensure the proper splicing signals are included once the vector has integrated. In one embodiment, the SD sequence includes about 15 bp upstream of the exon-intron boundary and about 300 bp downstream of the exon-intron boundary. These selected sequences can be analyzed using a splicing prediction algorithm to ensure that the intended splice sites are very strong and there are no other strong splice sites in the SA or SD. The frame of the about 15 bp exonic regions at the end of the SA and beginning of the SD do not necessarily need to match the frame of the first detectable marker, but these sequences should not contain any stop codons. The SA sequence, the SD sequence, poly-A sequences, recombinases and a FlipTrap cassette are further described in US20070101452, which is incorporated by reference in its entirety.

One or more methods for genomic editing in a target cell are provided, wherein the methods include introducing into the target cell a system that comprises (1) one or more donor sequences in one or more nucleic acid vectors, (2) a pair of gRNA for each donor sequence, the pair comprising a first gRNA and a second gRNA that flank the donor sequence, (3) one or more gRNA, each capable of binding a gene locus of interest in the target cell, and (4) a quantity of Cas nucleases (e.g., Cas9, Cas12a, Cas12b).

In some embodiments, the provided genomic editing methods are in vitro methods. In some embodiments, the provided genomic editing methods are ex vivo methods. In some embodiments, the provided genomic editing methods are in vivo methods.

Some embodiments of the genomic editing methods include the steps where: the first gRNA of a pair binds upstream of a donor sequence on its sense strand, and the second gRNA of the pair binds downstream of the donor sequence on its anti-sense strand. In some embodiments of the methods, the gRNA capable of binding a gene locus of interest in the target cell binds on the anti-sense strand.

Some embodiments of the methods for in vitro, ex vivo, or in vivo genomic editing in a target cell comprise: forming a first complex among a donor sequence, a first gRNA capable of binding upstream of the donor sequence, and a Cas nuclease; forming a second complex among the donor sequence, a second gRNA capable of binding downstream of the donor sequence, and a Cas nuclease; forming a third complex between a Cas nuclease and a third gRNA capable of binding a locus of interest in the target cell; and introducing the first, second, and third complexes to the target cell for genomic editing.

Other embodiments of the methods for genomic editing in a target cell comprise: forming a first complex between a donor sequence and a first gRNA capable of binding upstream of the donor sequence; forming a second complex between the donor sequence and a second gRNA capable of binding downstream of the donor sequence; forming a third complex between a Cas nuclease and a third gRNA capable of binding a locus of interest in the target cell; and introducing the first, second, and third complexes to the target cell for genomic editing.

Further embodiments of the methods for genomic editing in a target cell comprise: (1) mixing a donor sequence with a first gRNA and/or a second gRNA in the presence of a quantity of a Cas nuclease (e.g., Cas9), wherein the first gRNA and the second gRNA are capable of binding upstream and downstream of the donor sequence, respectively, thereby forming a first complex based on the donor sequence, the first gRNA, and the Cas nuclease, and/or a second complex based on the donor sequence, the second gRNA, and the Cas nuclease; (2) mixing a third gRNA with a Cas nuclease (e.g., Cas9), thereby forming a third complex; and (3) introducing into the target cell the first complex and/or the second complex, and the third complex, e.g., in an effective amount to induce non-homologous end joining repair or homology directed repair in the target cell.

In some embodiments, the donor sequence is in a plasmid, and the weight ratio in the mixing step of the donor sequence:the first (or second) gRNA:Cas9 nuclease is 2:1:8. In some embodiments, the weight ratio in the mixing step of the donor sequence:the first (or second) gRNA:Cas9 nuclease is 1:1:5, 1:1:4, 1:1:3, 1:1:2, or 1:1:1. In some embodiments, the weight ratio in the mixing step of the donor sequence:the first (or second) gRNA:Cas9 nuclease is 2:1:10, 2:1:9, 2:1:7, 2:1:6, 2:1:5, 2:1:4, 2:1:3, or 2:1:2.

In some embodiments, the donor sequence is linearized DNA, and the weight ratio in the mixing step of the donor sequence:the first (or second) gRNA:Cas9 nuclease is 1:1:8. In some embodiments, the weight ratio in the mixing step of the donor sequence:the first (or second) gRNA:Cas9 nuclease is 1:1:10, 1:1:9, 1:1:7, 1:1:6, 1:1:5, 1:1:4, 1:1:3, or 1:1:2. In some embodiments, the weight ratio in the mixing step of the linearized donor sequence:the first (or second) gRNA is 2:1, 3:1, 4:1, 5:1, or 1:2, 1:3, 1:4, 1:5.

In various embodiments, the mixing step includes a buffer of 20 mM HEPES, 150 mM KCl, and 1% sucrose at pH 7.5. In some embodiments, the mixing step includes mixing in a buffer containing 5-10, 10-20, 20-30, 30-40, or 40-50 mM HEPES, 100-150, 150-200, 200-250 or 250-300 mM KCl, and 0.5-1, 1-1.5, or 1.5-2% sucrose.

Further embodiments provide a method of genomic editing in a target cell to create a conditional allele or a fusion protein, the method comprising (1) introducing into the target cell a system including: a first gRNA, a second gRNA, a third gRNA, one or more RNA-guided nucleases, and a donor sequence in a nucleic acid vector, wherein the nucleic acid vector comprises a nucleic acid backbone sequence, a splice acceptor sequence, a splice donor sequence, a first recombination site pair (formed by a first recombination site and a second recombination site), a second recombination site pair (formed by a third recombination site and a fourth recombination site), and a polyadenylation sequence, and optionally further comprising a nucleic acid insertion sequence (e.g., a transposon sequence); and (2) introducing into the target cell one or more recombinases (e.g., Cre, Flp) creating a first recombination and optionally a second recombination event.

In various embodiments, the methods for genomic editing are suitable for use in any organism or cell line of choice. In various embodiments, the methods for integrating a FlipTrap cassette with the multi- (trio) gRNA system can be used in any organism or cell line that has introns. Established genetic organism systems for in vivo animal cell analysis in an intact animal, includes but is not limited to zebrafish, C. elegans, Drosophila melanogaster, medaka (rice fish), and mouse. Cell lines can also be used such as any animal or plant cell. Cultured animal cell lines such as embryonic stem (ES) cells can be used as the receiving cell. Mouse ES cells are an example of an established cultured cell line. Additionally, fertile animals can be recreated from mouse ES cells. In some embodiments, the methods for genomic editing can be used in human or in cells or tissues of human. In some embodiments, the methods for genomic editing are suitable for use in non-human organism. In some embodiments, the methods for genomic editing are suitable for use in non-human mammals.

Accordingly, an organism altered by one or more genomic editing methods disclosed herein is provided, wherein in various aspects, the organism is a non-human organism. In some aspects, the organism is non-human mammals.

Further embodiments are provided for scalable use of the trio gRNA system. In some aspects, a plurality of donor genes (e.g., two or more different expression cassettes) can be introduced into one or more loci of interest in the same batch, by using one or more gRNAs that target the one or more loci of interest. In other aspects, a quantity of a same donor gene can be introduced into two or more loci of interest, by using two or more gRNAs that target the two or more loci of interest, whereas the quantity of the donor gene can each be flanked by an identical pair of gRNAs. In some aspects, a plurality of donor genes (e.g., two or more different expression cassettes) are each flanked by a pair of gRNA, optionally further forming DNA:RNP complexes, and mixed with gRNA that targets a locus of interest. In some aspects, a plurality of donor genes (e.g., two or more different expression cassettes) can each be synthesized, or constructed in a vector, with an upstream sequence and a downstream sequence that are each recognizable by a gRNA, so that a pair of gRNA flanks a donor gene (e.g., an expression cassette), optionally further forming a DNA:RNP complex (with a Cas nuclease and the gRNA) at the upstream position and a DNA:RNP complex at the downstream position; and high-throughput assays can be performed by introducing each donor gene (flanked by a pair of gRNA or flanked by a pair of DNA:RNP complexes), to a locus of interest (via gRNA:Cas nuclease-targeted recombination) in a different cell, wherein different donor genes can share a pair of flanking gRNA of the same sequences.

### EXAMPLES

### Example 1. Zebrafish Maintenance and Strains

This study was carried out in accordance to the recommendations in the Guide for the Care and Use of Laboratory Animals. Adult fish were maintained as described. Wild-type embryos for CRISPR were obtained from AB/TL mix strains.

### Example 2. Guide RNA Design

The Inventors used the web program CHOPCHOP (chopchop.cbu.uib.no) to identify potential sgRNA target sequences. Genomic or plasmid sequences of 2 Kb were used to search for potential sgRNA targets. Target sequences were chosen that had no related potential off-target sites in the genome, exhibited between 40-60% GC content, and no mismatch sequence to the target. For sgRNAs to the donor sequence, the Inventors chose candidate sgRNAs that target flanking the tol2 sequences to ensure that the entire FlipTrap sequence would be integrated.

### Example 3. Generation of Guide RNA

Guide RNAs was generated with unique oligonucleotides containing the T7 RNA polymerase recognition site (GAAATTAATACGACTCACTATAGGG, SEQ ID NO:1), sgRNA target sequence, and an overlapping sequence (GTTTTAGAGCTAGAAATAGC, SEQ ID NO:2) with the tracrRNA. Exemplary sgRNA target sequence are available in Table 1. The unique sgRNA oligo was annealed to a universal oligo containing tracrRNA sequence (AAAAGCACCGACTCGGTGCCACTTTTTCAAGTTGATAACGGACTAGCCTTATTTT AACTTGCTATTTCTAGCTCTAAAAC, SEQ ID NO:3) and amplified by PCR. PCR product was used as template for in vitro transcription to generate guide RNAs. In vitro transcription was performed with 200 ng purified DNA template using the HiScribe™ T7 High Yield RNA Synthesis Kit (NEB) and purified using the RNeasy Mini Kit (Qiagen).

### Example 4. In Vitro Testing of sgRNA Cutting Efficiency

DNA template between 2-3 Kb was amplified using genomic DNA of the same genetic background as the embryos used for injections. The FlipTrap plasmid (sequence available through NCBI accession no. JN564735) was used as template for PCR amplification to generate DNA template. Primers for PCR amplification of the respective DNA templates used for in vitro sgRNA cutting efficiency test available in Table 2 (“gRNA cutting efficiency” or “HRMA” denotes the assay that primers were used for.). PCR product for in vitro sgRNA cutting test was purified using the QIAquick PCR Purification Kit (Qiagen). In vitro sgRNA cutting efficiency was performed with 100 ng of DNA template using the Guide-it™ sgRNA Screening Kit (Takara, Cat. No. 632639).

### Example 5. Preparation of DNA:RNP Complex and Embryo Injection

Lyophilized Cas9 protein (PNA Bio, Catalogue # CP01) was suspended in nuclease-free H₂O to final concentration of 1 mg/mL Cas9 protein in 20 mM HEPES, 150 mM KCl, 2% sucrose and 1 mM dithiothreitol (DTT) (pH 7.5) and stored at −80° C. To preassemble DNA:RNP complexes, the donor DNA (50 ng/μL plasmid or 25 ng/μL linear 4.2 Kb DNA fragment), was mixed with the various combinations of donor and target sgRNAs (25 ng/μL each), Cas9 protein (200 ng/μL) and 10% phenol red (5 mg/mL in 150 mM KCl; Sigma-Aldrich) and stored overnight at −20° C. prior to microinjections. Approximately, 2.3 nL of preassembled DNA:RNP complex was injected into the cytoplasm of one-cell stage embryos.

For linear donor DNA injections, the FlipTrap plasmid was digested with KpnI and NotI to release the vector backbone and gel purified to generate a linear DNA template of 4.17 Kb. DNA:RNP complexes containing linear DNA, donor sgRNAs and either Cas9 or dCas9 (PNA Bio, Catalogue #CD01) was preassembled separately from RNP complexes containing target sgRNA and Cas9 and stored at −20° C. overnight. Prior to microinjections, equal volume of donor DNA:RNP and target RNP complexes were mixed to bring the solution to a working solution that contained 25 ng/μL linear DNA, 200 ng/μL Cas9 protein, 25 ng/μL of each sgRNA and 10% phenol red.

### Example 6. High Resolution Melting Analysis

Genomic DNA was extracted from individual embryos at 5dpf using NaOH extraction protocol as follows: Single embryos were incubated in 50 mM NaOH for 10 minutes at 95° C. and cooled to 4° C.

A 1/10th volume of 1M Tris-HCl, pH 8.0 was added to samples to neutralized solution. Primers were designed to span ˜200 bp of the sgRNA cutting site. Sequence of primers available in Table 2. LightCycler 480 mastermix (Roche Life Science) was used to perform PCR on a BioRad C1000 Thermal Cycler with the following conditions: 95° C. −3 minute denaturation, 48 cycles of 95° C. 30 seconds denaturation, 58° C. 30 seconds annealing, and 72° C. 30 seconds extension, 72° C. 2 minutes extension, with melting curve of 65° C.-95° C. increment of 0.2° C. for 0.05 second.

### Example 7. Trio sgRNA Strategy

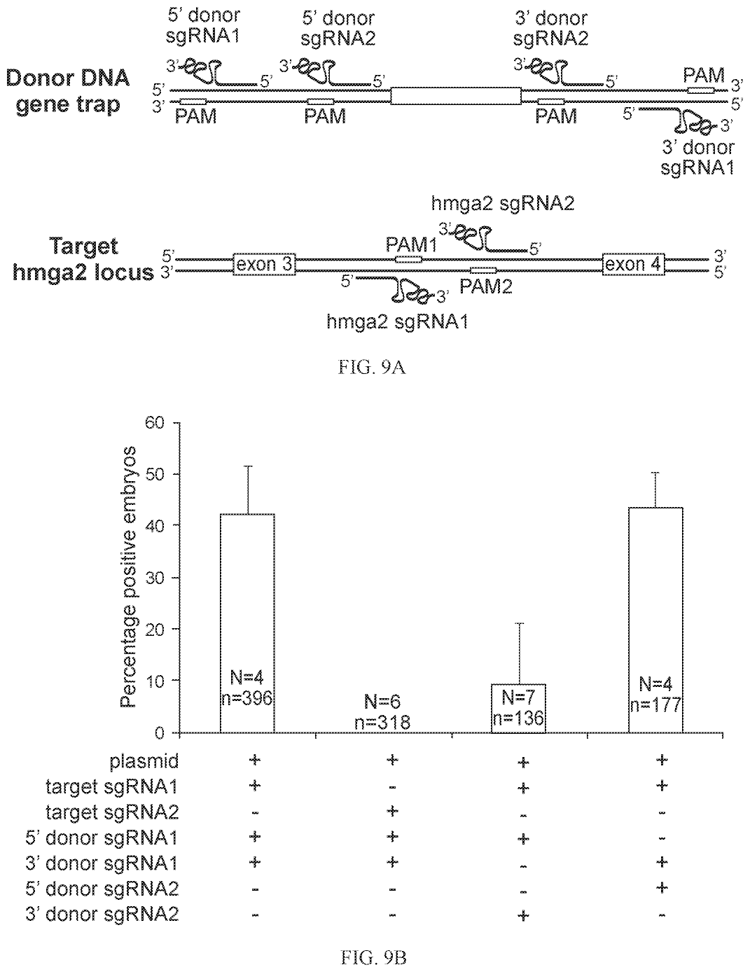

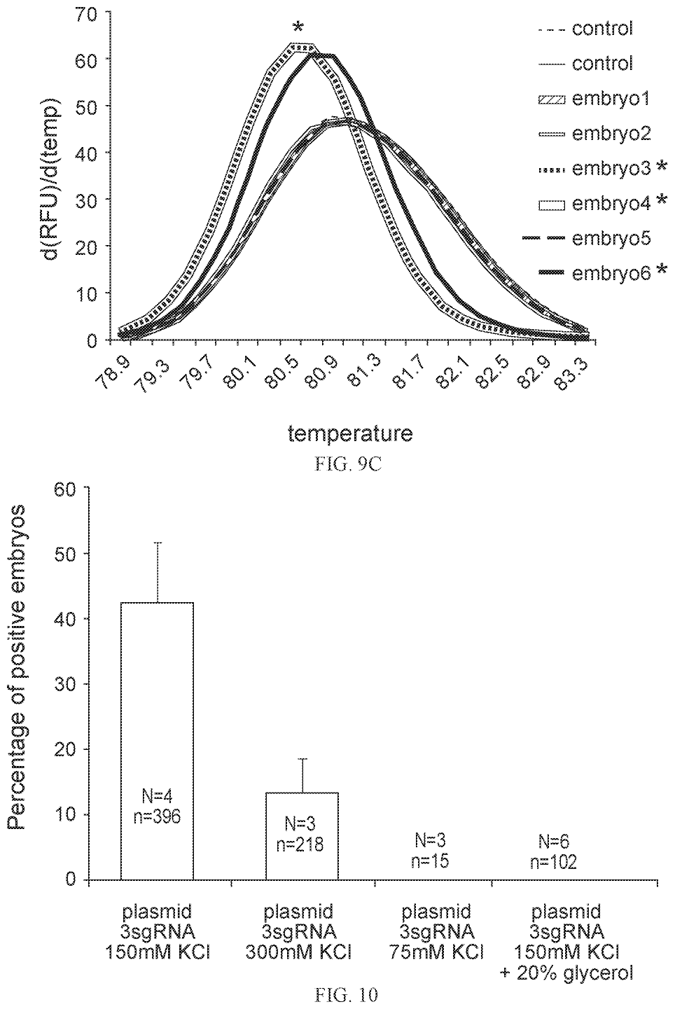

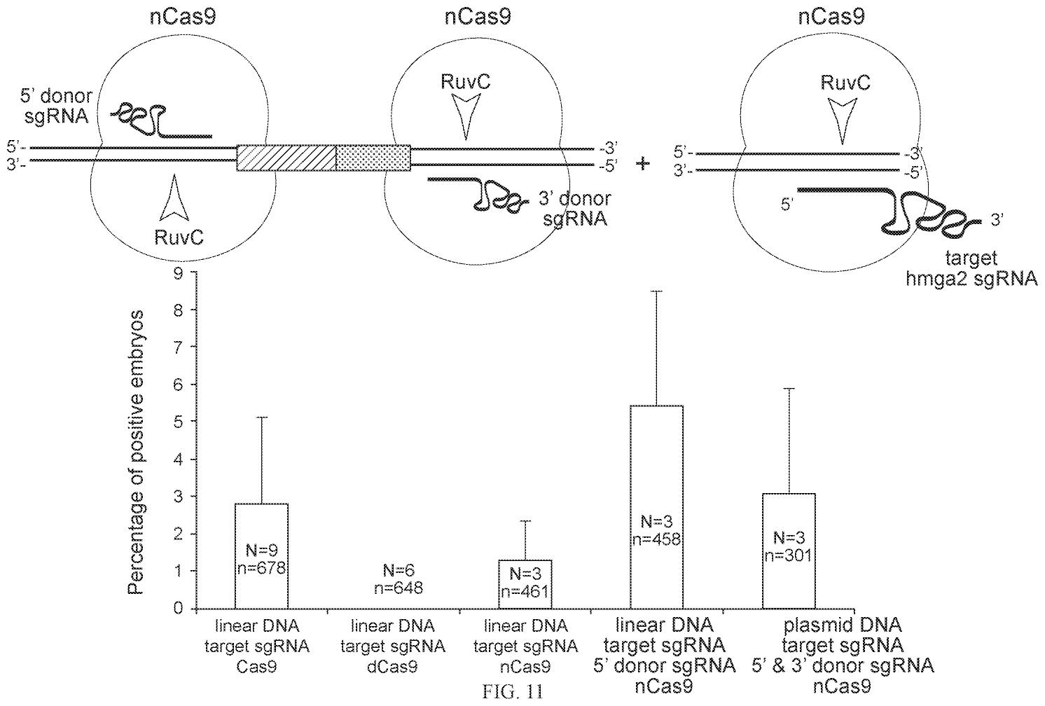

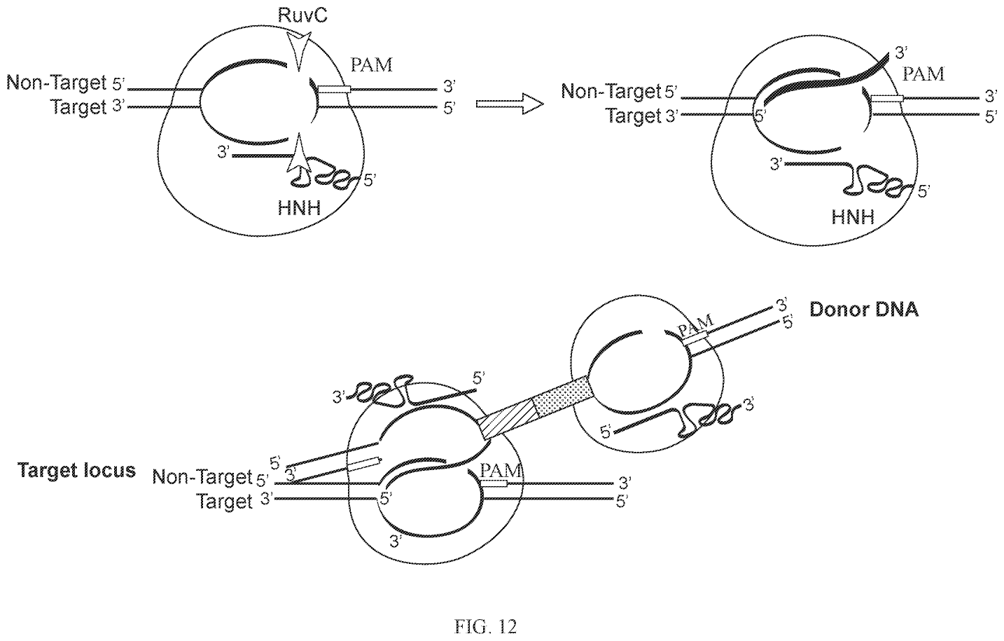

The Inventors used CRISPR/Cas9 to integrate the FlipTrap sequence into specific loci within the zebrafish genome. The inventors reasoned that integration of FlipTrap sequence would be more efficient if the plasmid was linearized and the plasmid backbone was excised during the integration process. To achieve both linearization and excision of the plasmid backbone, the Inventors designed an integration strategy that uses a pair of donor sgRNAs to bind and cleave the 5′ and 3′ ends of the donor sequence (sequence within the plasmid to be integrated, 4.2 Kb fragment) and a single sgRNA to target the locus of interest (FIG. 14A). The Inventors tested the trio sgRNA strategy on a locus that the Inventors previously characterized from a gene trap screen, high mobility group AT-hook 2 (hmga2) (FIG. 14A). The gene trap allele, Gt(hmga2-citrine)^{ct29a}, contains the FlipTrap sequence integrated into the third intron between exons 3 and 4 of the hmga2 locus, creating a Citrine fusion protein with Citrine 3′ of the three AT-hook DNA binding domains. To target the hmga2 locus with the trio sgRNA approach, the Inventors designed two targeting sgRNAs to intron 3 of hmga2 (Table 1), one binding to the sense strand (hmga2-sgRNA1) and one binding to the anti-sense strand (hmga2-sgRNA2). To enable cleavage and insertion of the donor DNA, two pairs of donor sgRNAs were designed to target the 5′ and 3′ ends of the donor sequence within the FlipTrap plasmid, designated as FT-5 or FT-3, respectively (Table 1). All 5′ donor sgRNAs bound the sense strand (therefore targeted the anti-sense strand), while one of the two 3′ donor sgRNAs bound the anti-sense strand (therefore targeted the sense-strand) of the FlipTrap sequence (FIG. 9A).

The Inventors performed in vitro screening to test the cutting efficiency of the donor and targeting sgRNAs. For template DNA, the Inventors used the FlipTrap plasmid and genomic DNA to test the cutting efficiency of the donor and targeting sgRNAs, respectively. All donor and targeting sgRNAs cut their respective template with equal efficiency (FIG. 14B). Based on the in vitro cutting efficiency, the Inventors proceeded to use donor and targeting sgRNAs for in vivo integration of the FlipTrap sequence into the zebrafish genome.

### Example 8. Trio sgRNA Increases Efficiency of Donor DNA Integration

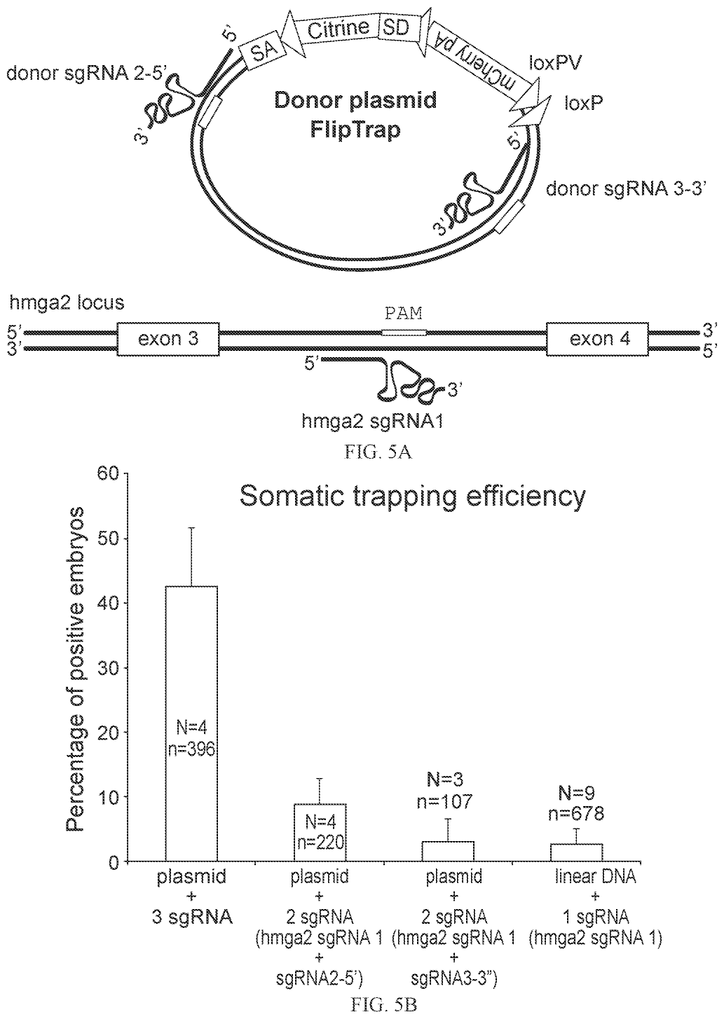

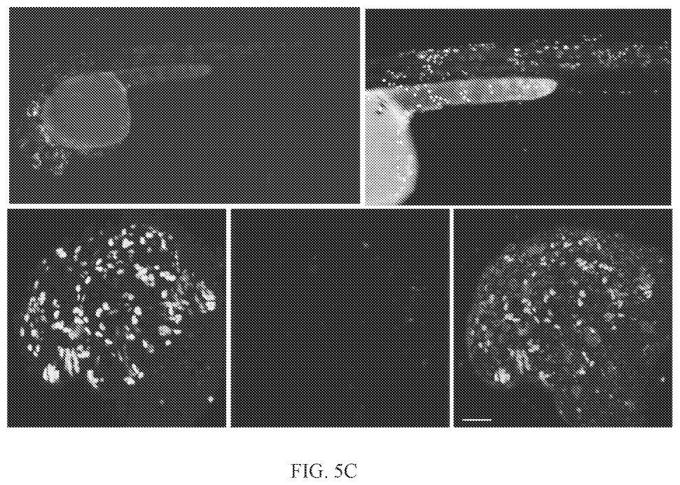

The Inventors tested the efficiency of the trio sgRNA approach by injecting preassembled (plasmid) donor DNA, sgRNAs and Cas9 protein into one-cell-stage zebrafish embryo. The Inventors used the ribonuclear protein (RNP) approach to reduce off-target effects. The Inventors' injection solutions contained preassembled DNA:RNP complex of donor DNA, donor and target sgRNAs and Cas9 protein. The Inventors used the hmga2-sgRNA1 to target the donor FlipTrap sequence into the hmga2 locus. For donor sgRNA, the Inventors used the FT5-1 and FT3-1 donor pairs (FIG. 9A). Injection of DNA:RNP complexes that contained all three sgRNAs (donor pair and single target sgRNAs) resulted in 42.5% (N=4, n=396) of the injected embryos exhibiting Citrine expression localized to the nucleus (FIGS. 5B and 5C). The nuclear localization of the Citrine reporter is consistent with integration of the FlipTrap sequence into the hmga2 locus as Hmga2 protein localizes to the nucleus of cells. Injection of DNA:RNP complexes that contained only one donor sgRNA and the target sgRNA (hmga2-sgRNA1) resulted in 8.9% and 3% of the embryos expressing Citrine in the nucleus of cells for the 5′ and 3′ donor sgRNAs, respectively (FIG. 5B). These results indicate that the three sgRNAs synergize to increase the integration efficiency of donor DNA as the Inventors observed a 13- and 8-fold increase in integration efficiency over the use of two sgRNAs.

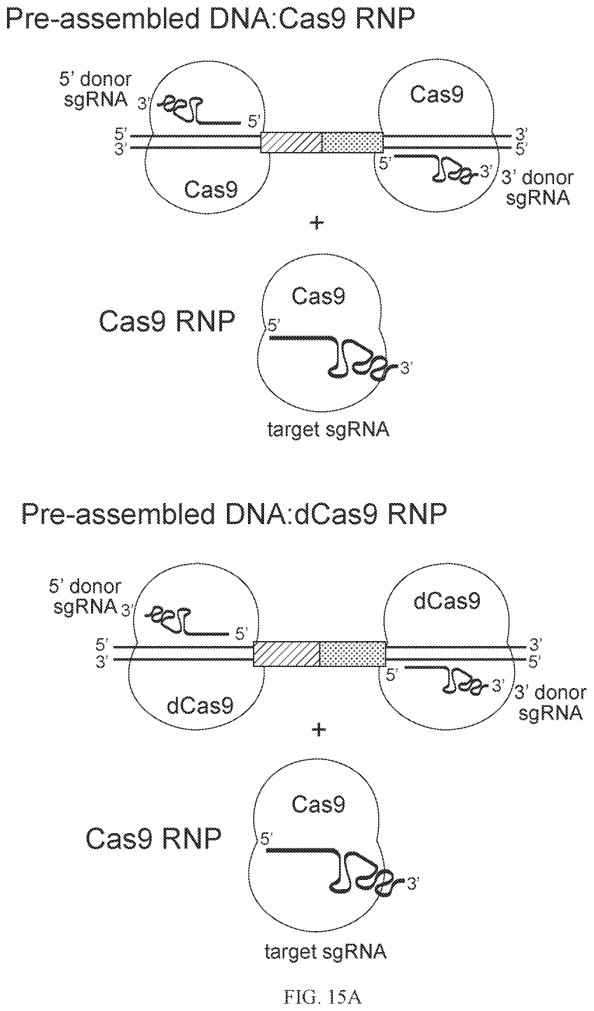

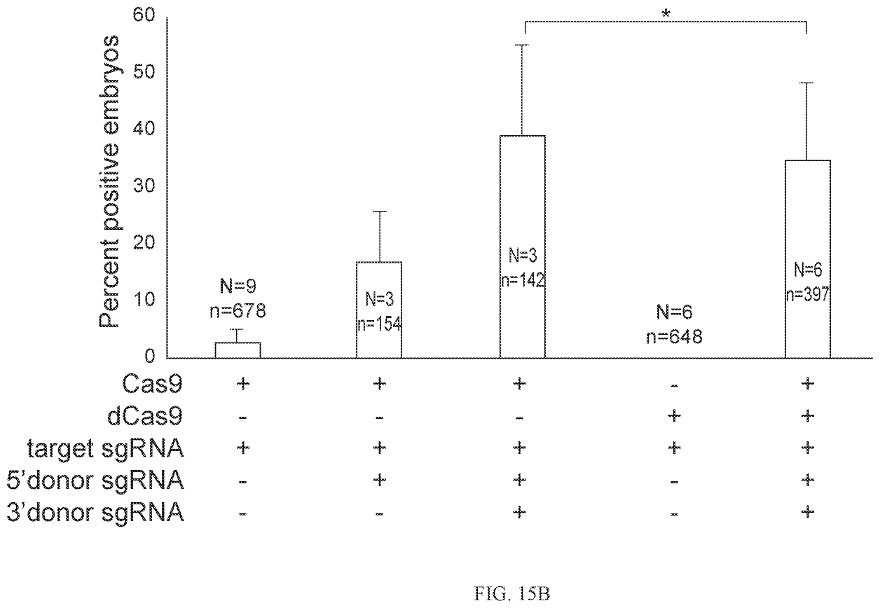

Next, the Inventors asked whether the same synergy in DNA integration occurs with the trio sgRNA approach using linearized donor DNA. Injection of DNA:RNP complexes containing linear DNA (4.173 Kb fragment containing only the FlipTrap sequence) and a single target sgRNA (no donor sgRNAs) resulted in 2.8% of the embryos exhibiting Citrine expression (FIG. 15B). However, injections of DNA:RNP complexes containing linear DNA and two (one donor and one target) or three (two donors and one target) sgRNAs resulted in 16.8% and 39.1% of embryos expressing Citrine in the nucleus, respectively (FIG. 15B). The 13-fold increase in DNA integration observed with three sgRNA in the DNA:RNP complex solution is similar between linear and plasmid DNA.

The Inventors then asked whether the increase in DNA integration is dependent on the ability of Cas9 to cleave the donor DNA. To address this question, the Inventors pre-assembled the donor DNA:RNP using nuclease inactive Cas9 (dCas9), and pre-assembled the target RNP complexes using wildtype Cas9(FIG. 15A). The separate pre-assembly of donor DNA:RNP and target RNP complexes was need to ensure that the targeting RNP complex contained wildtype Cas9, as microinjection of linear DNA and targeting RNP complexes containing dCas9 resulted in no detectable integration of the linear DNA (FIG. 15B). However, donor DNA:RNP complexes containing linear DNA, both donor sgRNAs and dCas9 when mixed with targeting RNP complexes containing wildtype Cas9 resulted in efficient DNA integration with 34.6% of embryos expressing Citrine in the nucleus (FIG. 15B). The rate of integration with linear DNA:RNP complexes containing dCas9 showed no statistical differences when compared to linear DNA:RNP complexes that contained wildtype Cas9, indicating that the presence of the DNA:RNP complex is sufficient for the increase in DNA integration observed with the trio sgRNA approach. Therefore, the increase in DNA integration does not appear to be dependent on the ability of Cas9 to cleave the donor DNA (at least for linearized donor DNA), but the presence or pre-assembly with Cas9 or dCas9 for donor DNA (in the trio sgRNA system) increased the DNA integration.

### Example 9. NHEJ-Mediated Integration of Donor DNA

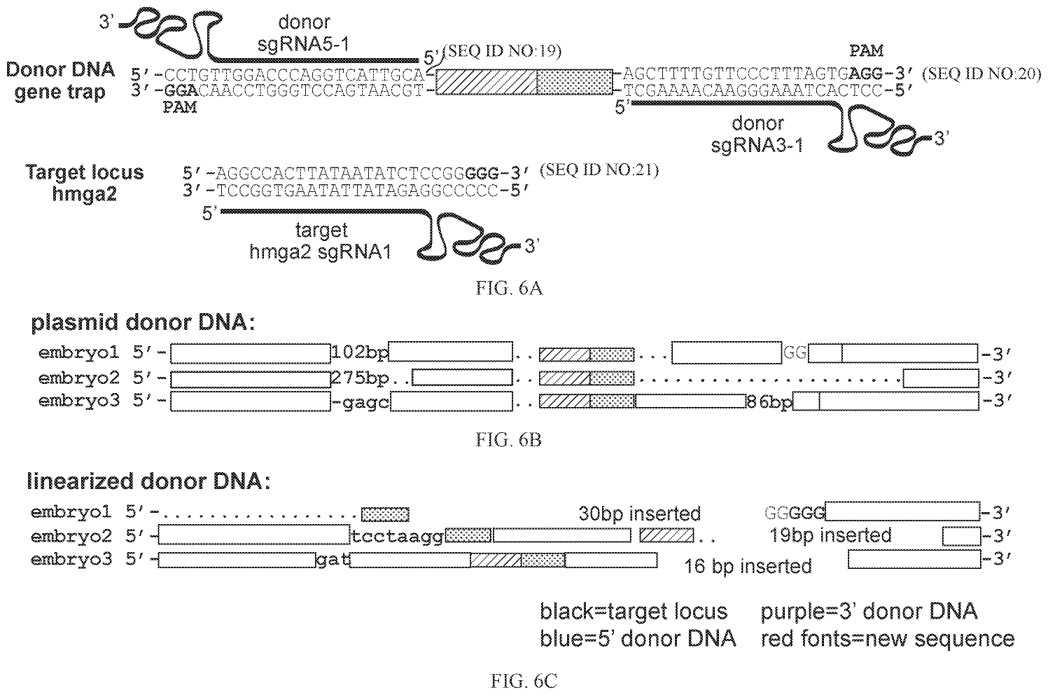

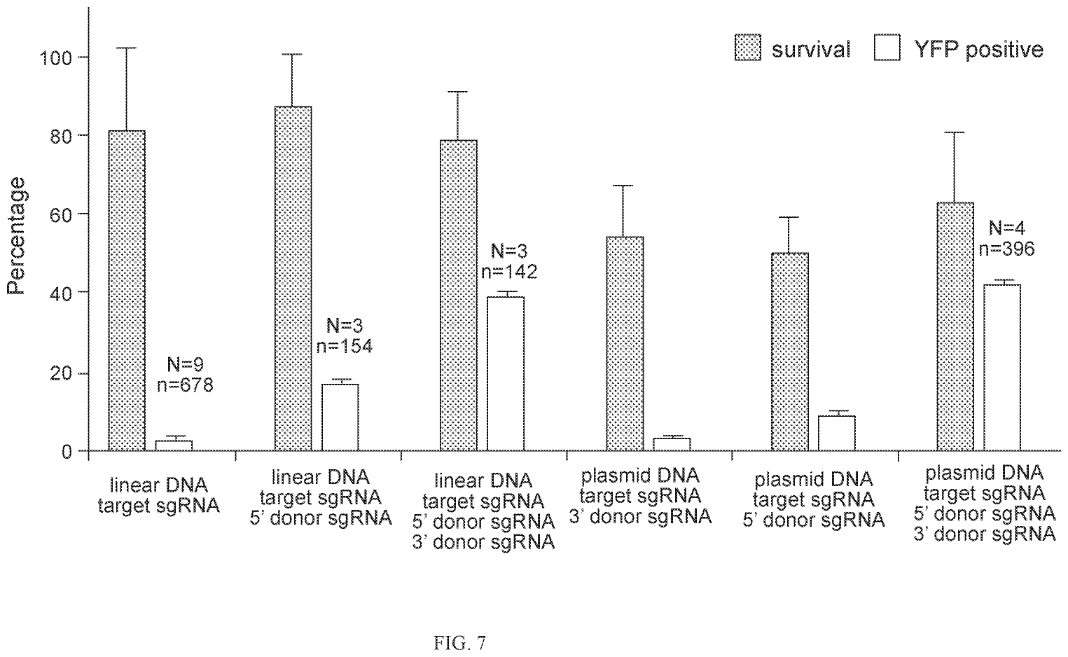

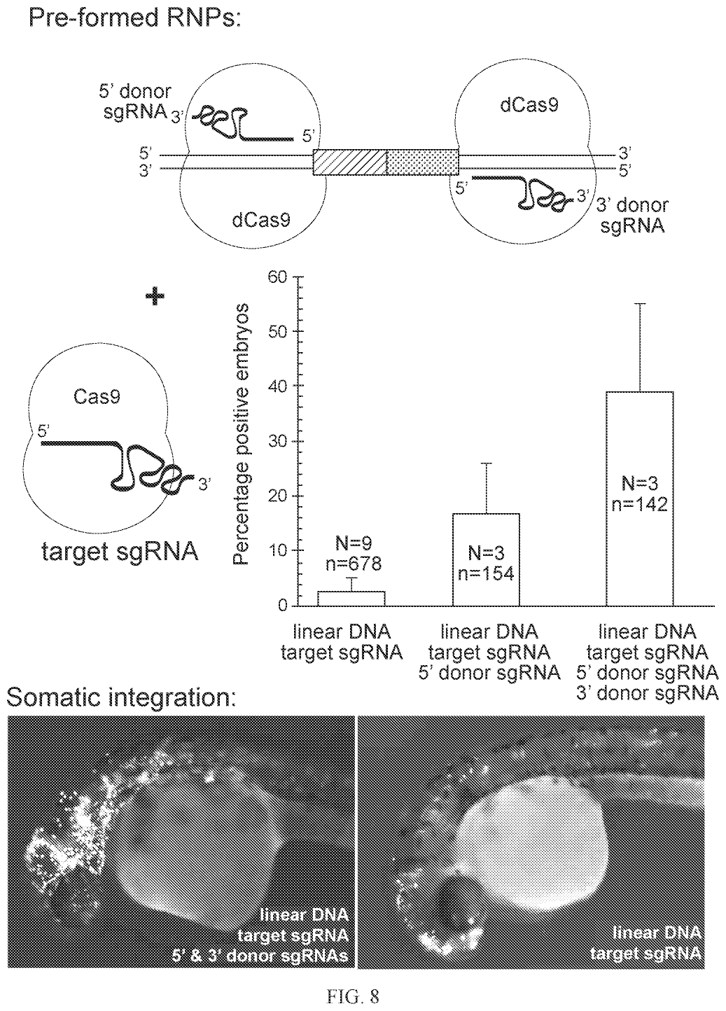

As the donor DNA does not contain homology sequence to the target locus, the Inventors examined the integration sites within individual embryos to determine the nature of the DNA repair. Sequencing of genomic DNA from individual embryos injected with DNA:RNP complexes containing plasmid DNA and the three sgRNAs showed precise insertion 5 nucleotides upstream of the PAM sequence in some cases (FIG. 6B, embryo 1 and 2) and small indels in others (FIG. 6B, embryo 3) for the target locus. However, the donor sequence showed a wide range of possibilities for insertion events. In some cases, insertions of the donor sequence occurred a few hundred nucleotides (e.g., 102 bp, or 275 bp) upstream of the 5′ donor sgRNA binding sites (FIG. 6B, embryos 1 and 2), indicating that the donor sgRNA may not be cutting the donor DNA at the binding sites. In other cases, insertion of the donor sequence occurred within the binding sites of the 5′ and 3′ donor sgRNA (FIG. 6B, embryo 3). Together, these results indicates that the trio sgRNA approach leads to NHEJ-mediated repair after integration of the donor DNA.

Interestingly, sequencing from embryos injected with preassembled DNA:RNP complexes containing linear DNA, and only the targeting sgRNA with Cas9 protein, shows aberrant recombination with inversions and deletions of the donor DNA (FIG. 6C). In one case, sequencing could not identify the 5′ end of the donor DNA (FIG. 6C, embryo 1), while in another case, citrine sequence had integrated into the reverse orientation (FIG. 6C, embryo 2). These results indicate that the presence of the donor RNP complex may aide in suppressing aberrant recombination of the donor DNA.

### Example 10. Directionality of sgRNA Affects Donor DNA Integration Efficiency

The difference in DNA integration efficiency observed between the 5′ (FT5-1) and 3′ (FT3-1) donor sgRNAs when paired with the target sgRNA (hmga2-sgRNA1) (FIG. 5B) led us to examine the directionality of the sgRNA as the Inventors noted that the 5′ and 3′ donor sgRNAs was complementary to the sense and anti-sense stands of the FlipTrap sequence, respectively, while the targeting sgRNA bound to the anti-sense strand (FIG. 14A). Cas9 has been reported to interact asymmetrically with the target DNA. Additionally, the efficient integration and suppression of aberrant recombination of linear DNA when preassembled with dCas9 indicate a potential interaction between the donor DNA:RNP and the targeting RNP complexes. Combined, this led us to hypothesize that the high efficiency observed in the trio sgRNA approach may come from interactions between the DNA:RNP complexes that are dependent on the directionality of the donor and target sgRNAs.

To test this hypothesis, the Inventors formed DNA:RNP complexes with different combinations of donor and targeting sgRNAs that bound to either the sense or anti-sense strand of the donor and target DNA (FIG. 9A). First, the Inventors compared the integration efficiency of target sgRNAs that bound to either sense or anti-sense strand, while keeping the donor sgRNAs in the initial 5′ sense-binding and 3′ anti-sense-binding configuration. DNA:RNP complexes containing target sgRNA that bound to the anti-sense strand (hmga2-sgRNA1) exhibited high DNA integration efficiency (FIG. 9B, 42.5%). In contrast, injections of DNA:RNP complexes containing target sgRNA that bound to the sense strand (hmga2-sgRNA2) exhibited no detectable DNA integration. The absence of DNA integration was not due to the inability of the hmga2-sgRNA2 to target the hmga2 locus, as indels were detected from individual embryos injected with DNA:RNP complexes containing the hmga2-sgRNA2 (FIG. 9C). Next, the Inventors compared the contribution of strandedness of donor sgRNA on integration efficiency. DNA:RNP complexes containing donor sgRNAs that bound to the sense strand on both the 5′ and 3′ ends with the target sgRNA bound to the anti-sense strand resulted in 10% of the injected embryos exhibiting positive Citrine expression. This is a 4-fold reduction in DNA integration efficiency as compared to injections of DNA:RNP complexes containing 5′ and 3′ donor sgRNAs that bound to the sense strand and anti-sense strand, respectively (FIG. 9B). Finally, DNA:RNP complexes containing a second 5′ donor sgRNA (FT5-2) that also binds the sense-strand of the donor DNA, with a 3′ anti-sense-binding donor sgRNA and an anti-sense-binding targeting sgRNA (hmga2-sgRNA1) resulted in high DNA integration efficiency (FIG. 9B, 43.9%). Additional loci have been successfully targeted with this combination of donor sgRNA binding to the sense and anti-sense on the 5′ and 3′ end of the donor DNA, respectively, and the target sgRNA binding to the anti-sense strand of the individual locus (Table 1; fn-1, fn-2 and col4a1 are loci of interest in host cells that were edited with the trio sgRNA system.). Combined, these results indicate that integration efficiency of donor DNA is dependent on the directionality of sgRNA to both the donor DNA and targeting locus, with the optimal combination of the 5′ donor sgRNA binding to the sense-strand and the 3′ donor and target sgRNA binding to the anti-sense strand.

TABLE 1
The targeting sequence (crRNA region) of sgRNA

| | | Targeting |
|---|---|---|
| sgRNA | | (Homologous To) |
| target | crRNA Sequence | Strand |
| FT5-1 | TGCAATGACCTGGGTCCAAC (SEQ ID NO: 4) | anti-sense |
| FT5-2 | TGTGTGGAACAGAGTGGATA (SEQ ID NO: 5) | anti-sense |
| FT3-1 | AGCTTTTGTTCCCTTTAGTG (SEQ ID NO: 6) | sense |
| FT3-2 | GGCGGCCGCTCTAGAACTAG (SEQ ID NO: 7) | anti-sense |
| hmga2-1 | GGCCACTTATAATATCTCCGG (SEQ ID NO: 8) | sense |
| hmga2-2 | AGGTGTTTACTTGTCTGCAG (SEQ ID NO: 9) | anti-sense |
| fn-1 | AGACGCAAATTGTTTTATAA (SEQ ID NO: 10) | sense |
| fn-2 | CAAGCAAACTACTGCGTACG (SEQ ID NO: 11) | sense |
| col4a1 | GCATAACAAGGGAATCTACA (SEQ ID NO: 12) | sense |

TABLE 2
Primers for DNA amplification

| Primers | Sequence |
|---|---|
| hmga2_intron3_F3 (gRNA cutting efficiency) | tacatggacaccaactaagacaata (SEQ ID NO: 13) |
| hmga2_intron3_R3 (gRNA cutting efficiency) | ccaaacaaataacatacaatgtgaa (SEQ ID NO: 14) |
| FlipTrap-F (gRNA cutting efficiency) | gtactggcattagattgtctgtctt (SEQ ID NO: 15) |
| FlipTrap-R (gRNA cutting efficiency) | ttataatttccctaatttccaggtc (SEQ ID NO: 16) |
| hmga2-intron3-F (HRMA) | ggcccttagattcgtcctaa (SEQ ID NO: 17) |
| hmga2-intron3-R (HRMA) | cctacgacaaacgctgagat (SEQ ID NO: 18) |

The Inventors have found that the trio sgRNAs synergize to increase the efficiency of large donor DNA integration via non-homology end joining (NHEJ) repair. The efficiency of donor DNA targeting depends on the directionality of the donor and target sgRNAs. Highest efficiency is observed with sgRNAs targeting the 5′ of the donor DNA on the anti-sense strand and the target locus on the sense strand. Furthermore, the multiple sgRNA directed towards donor DNA suppress aberrant recombination of the donor DNA. Together, this enhanced approach offers dramatically improved integration and scalability into a specified locus as targeting different loci requires the change of only a single sgRNA.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof. Many variations and alternative elements have been disclosed in embodiments of the present invention. Still further variations and alternate elements will be apparent to one of skill in the art. Among these variations, without limitation, are the compositions for, and methods of, genetic editing, in vivo methods associated with genetic editing, compositions of cells generated by the aforementioned techniques, treatment of diseases and/or conditions that relate to the teachings of the invention, techniques and composition and use of solutions used therein, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventor for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

The embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A system for integrating a donor sequence into genome of a target cell, comprising:
a first guide RNA (gRNA);
a second gRNA; and
a third gRNA,
wherein the first and second gRNA flank the donor sequence and are independently capable of guiding a nuclease to a respective region of the donor sequence flanked by the first and second gRNA, and
wherein the third gRNA binds a locus of interest in the genome of the target cell and is capable of guiding a nuclease thereto.

2. The system of claim 1, wherein the donor sequence has a sense strand and an anti-sense strand, and the first gRNA is single-stranded guide RNA (sgRNA) and binds upstream of the donor sequence on the sense strand.

3. The system of claim 1, wherein the locus of interest in the genome of the target cell has a sense strand and an anti-sense strand, and the third gRNA is sgRNA and binds the locus of interest on the anti-sense strand in the genome of the target cell.

4. The system of claim 1, wherein the donor sequence has a sense strand and an anti-sense strand, the locus of interest has a sense strand and an anti-sense strand, and wherein the first gRNA is sgRNA and binds upstream of the donor sequence on the sense strand, the third gRNA is sgRNA and binds the locus of interest on the anti-sense strand in the genome of the target cell, and the second gRNA is sgRNA and binds downstream of the donor sequence on the anti-sense strand.

5. The system of claim 2, further comprising the donor sequence, one or more nucleases, or a combination of the donor sequence and the one or more nucleases.

6. The system of claim 5, comprising the donor sequence and one or more Cas nucleases, wherein the first gRNA and a first of the Cas nucleases form a complex with the donor sequence; the second gRNA and a second of the Cas nucleases form a complex with the donor sequence; the third gRNA and a third of the Cas nucleases form a complex; or a combination of forming any two or three of the complexes.

7. The system of claim 1, further comprising the donor sequence in a nucleic acid vector, wherein the nucleic acid vector comprises:
a nucleic acid backbone sequence,
a splice acceptor sequence,
a splice donor sequence,
a first recombination site and a second recombination site forming a first recombination site pair capable of being recognized by a first recombinase, wherein the first and second recombination sites are in opposite orientations,
a third recombination site and a fourth recombination site forming a second recombination site pair capable of being recognized by the first recombinase or a second recombinase, wherein the third and fourth recombination sites are in opposite orientations, and wherein the second recombination site pair flanks the second recombination site, but does not flank the first recombination site, and
a polyadenylation sequence;
wherein the first gRNA binds upstream of the first recombination site, and the second gRNA binds downstream of the fourth recombination site.

8. The system of claim 7, wherein the nucleic acid vector further comprises a nucleic acid insertion sequence selected from the group consisting of a transposon sequence, a viral sequence, and a homologous recombination sequence, and optionally further comprises a polyadenylation sequence which is not effective in the reverse orientation.

9. The system of claim 7, wherein the first recombination site pair is heterotypic with respect to the second recombination site pair, and the first recombinase and the second recombinase are selected from Cre or Flp.

10. The system of claim 7, wherein the nucleic acid vector further comprises one or more detectable marker sequences, each independently encoding a visible marker or a selectable marker; wherein the nucleic acid vector comprises two nucleic acid insertion sequences, the two nucleic acid insertion sequences are two transposable elements, wherein the splice acceptor sequence, the splice donor sequence, the first and second recombination sites, and the third and fourth recombination sites, and the one or more detectable marker sequences are internal to the two transposable elements; and wherein the first gRNA binds upstream of a first of the two transposable elements, and the second gRNA binds downstream of a second of the two transposable elements.

11. The system of claim 10, wherein the visible marker is a fluorescent protein, or a chromogenic enzyme.

12. A method of genomic editing in a target cell, comprising:
introducing into the target cell a system, the system comprising
a nucleic acid vector comprising a donor sequence,
a pair of a first guide RNA (gRNA) and a second gRNA, wherein the first gRNA comprises a sequence capable of binding upstream of the donor sequence and the second gRNA comprises a sequence capable of binding downstream of the donor sequence,
a third gRNA which comprises a sequence capable of binding a locus of interest in genome of the target cell, and
one or more RNA-guided nucleases derived from clustered regularly interspaced short palindromic repeat (CRISPR)-associated (Cas) system.

13. The method of claim 12, wherein the nucleic acid vector is a plasmid.

14. The method of claim 12, wherein the nucleic acid vector is a linearized DNA.

15. The method of claim 12, wherein the donor sequence has a sense strand and an anti-sense strand, the locus of interest in the genome of the target cell has a sense strand and an anti-sense strand, and wherein the first gRNA is single-stranded guide RNA (sgRNA) and binds the upstream of the donor sequence on the sense strand, and the third gRNA is sgRNA and binds the locus of interest on the anti-sense strand in the genome of the target cell.

16. The method of claim 12, wherein the one or more RNA-guided nucleases comprise a Cas9 nuclease selected from wild-type Cas9 nuclease, nicking Cas9 nuclease (nCas9), deactivated Cas9 (dCas9), or a combination thereof.

17. The method of claim 16, wherein the first gRNA and a first Cas9 nuclease form a first complex with the donor sequence before contacting the target cell;
the second gRNA and a second Cas9 nuclease form a second complex with the donor sequence before contacting the target cell;
the third gRNA and a third Cas9 nuclease selected from wild-type Cas9 nuclease or nCas9 nuclease form a third complex, before contacting the target cell; or
the first, second, and third complexes are formed before contacting the target cell.

18. The method of claim 12, wherein the one or more RNA-guided nucleases comprise a Cas9 nuclease, the donor sequence is at least 200 bp long, and the method results in integration of the donor sequence into the locus of interest in the genome of the target cell with an efficiency of at least 40%, 30-40%, 20-30%, or 10-20%.

19. The method of claim 12, wherein the introducing comprises microinjecting into the target cell or transfecting the target cell.

20. A method of genomic editing in a target cell to create a conditional allele or a fusion protein, the method comprising:
introducing into the target cell a system of claim 7, and
introducing into the target cell one or more recombinases creating a first recombination and optionally a second recombination event.

21. The method of claim 20, wherein the one or more recombinases are encoded genomically in the target cell.

22. The method of claim 20, wherein the one or more recombinases are introduced by transfection.

23. The method of claim 20, wherein the nucleic acid vector of the system encodes a visible marker or a selectable marker, and the locus of interest in the target cell is an intron within a genomic sequence encoding a protein, thereby the method creating a fusion protein wherein the visible marker or the selectable marker tags the protein in the target cell.

24. A quantity of cells made by the method of claim 12.

25. An organism altered by the method of claim 12.


SEQUENCE LISTING (21 sequences)

SEQ ID NO: 1 - DNA, 25 bp, Artificial Sequence
  synthetic construct
gaaattaatacgactcactataggg

SEQ ID NO: 2 - DNA, 20 bp, Artificial Sequence
  synthetic construct
gttttagagctagaaatagc

SEQ ID NO: 3 - DNA, 80 bp, Artificial Sequence
  synthetic construct
aaaagcaccgactcggtgccactttttcaagttgataacggactagccttattttaacttgctatttctagctctaaaac

SEQ ID NO: 4 - DNA, 20 bp, Artificial Sequence
  synthetic construct
tgcaatgacctgggtccaac

SEQ ID NO: 5 - DNA, 20 bp, Artificial Sequence
  synthetic construct
tgtgtggaacagagtggata

SEQ ID NO: 6 - DNA, 20 bp, Artificial Sequence
  synthetic construct
agcttttgttccctttagtg

SEQ ID NO: 7 - DNA, 20 bp, Artificial Sequence
  synthetic construct
ggcggccgctctagaactag

SEQ ID NO: 8 - DNA, 21 bp, Artificial Sequence
  synthetic construct
ggccacttataatatctccgg

SEQ ID NO: 9 - DNA, 20 bp, Artificial Sequence
  synthetic construct
aggtgtttacttgtctgcag

SEQ ID NO: 10 - DNA, 20 bp, Artificial Sequence
  synthetic construct
agacgcaaattgttttataa

SEQ ID NO: 11 - DNA, 20 bp, Artificial Sequence
  synthetic construct
caagcaaactactgcgtacg

SEQ ID NO: 12 - DNA, 20 bp, Artificial Sequence
  synthetic construct
gcataacaagggaatctaca

SEQ ID NO: 13 - DNA, 25 bp, Artificial Sequence
  synthetic construct
tacatggacaccaactaagacaata

SEQ ID NO: 14 - DNA, 25 bp, Artificial Sequence
  synthetic construct
ccaaacaaataacatacaatgtgaa

SEQ ID NO: 15 - DNA, 25 bp, Artificial Sequence
  synthetic construct
gtactggcattagattgtctgtctt

SEQ ID NO: 16 - DNA, 25 bp, Artificial Sequence
  synthetic construct
ttataatttccctaatttccaggtc

SEQ ID NO: 17 - DNA, 20 bp, Artificial Sequence
  synthetic construct
ggcccttagattcgtcctaa

SEQ ID NO: 18 - DNA, 20 bp, Artificial Sequence
  synthetic construct
cctacgacaaacgctgagat

SEQ ID NO: 19 - DNA, 23 bp, Artificial Sequence
  sythetic construct
cctgttggacccaggtcattgca

SEQ ID NO: 20 - DNA, 23 bp, Artificial Sequence
  synthetic construct
agcttttgttccctttagtgagg

SEQ ID NO: 21 - DNA, 25 bp, Artificial Sequence
  synthetic construct
aggccacttataatatctccggggg